(19) United States
(12) Patent Application Publication
(10) Pub. No.: US 2015/0093291 A1
(43) Pub. Date: Apr. 2, 2015

(54) COMPETITIVE BINDING DENDRIMER-BASED SYSTEM FOR ANALYTE DETECTION

(71) Applicant: RECEPTORS LLC, Chaska, MN (US)

(72) Inventors: Christina Thomas, Eden Prairie, MN (US); Robert E. Carlson, Minnetonka, MN (US)

(73) Assignee: RECEPTORS LLC, Chaska, MN (US)

(21) Appl. No.: 14/238,348

(22) PCT Filed: Aug. 13, 2012
(86) PCT No.: PCT/US12/50595

§ 371 Date: May 23, 2014

Related U.S. Application Data
(60) Provisional application No. 61/523,464, filed on Aug. 15, 2011.
(60) Provisional application No. 61/647,662, filed on May 16, 2012.

(51) Int. Cl. G01N 33/66; G01N 29/02; G01N 33/543

(52) U.S. Cl. CPC G01N 33/66; G01N 33/54373; G01N 33/54353; G01N 29/022
USPC 422/69; 436/501

(57) ABSTRACT

A sensitive, precise detector system for physiological analytes uses a novel system. The system comprises an immobilized polyol on a surface. Reversibly coupled to the polyol or analyte is a dendrimer structure. In the system, a signal is triggered by the dendrimer structure when in a competitive environment with an analyte at the surface. In one embodiment, the system is an implantable sensor for use by diabetic patients. The sensing system can produce a consistent, measurable response while functioning under biologically relevant conditions. The sensing system requires the interaction of two components: 1) a competitive agent/signaling component, a dendrimer-boronic acid construct (DBA) and 2) a binding environment for a glucose-competitive DBA competition, which is an immobilized monosaccharide mimic (iDIOL).

### BACKGROUND

In the management of chronic and acute disease, the measurement of a particular physiological analyte can be important. Similarly, in the operation of a variety of conventional chemical systems the measurement of a particular analyte can be an important process control parameter. The occurrence of an abnormal variation in concentration of a variety of analytes such as potassium, glucose, calcium, etc., in a patient's physiological chemical system can require immediate hospitalization or if left untreated the patient can suffer severe problems.

In order to prevent these difficulties, the need to obtain real-time measurements of chemical, biological or physiological analytes is important to prevent large abnormal changes. Early detection of an abnormal tendency, can be dealt with or treated, in order to prevent serious problems. Additionally, in hospitalized patients, the rapid or real-time measurement of physiological analytes can also be important in maintaining homeostasis and avoiding critical care situations involving intensive care units or expensive treatment protocols.

Currently, levels of chemical, biological or physiological analytes are measured using automated or manual laboratory systems. Conventional chemical samples can be obtained using routine techniques while biological samples can be obtained from veinous blood either using commonly available Vacutainer® systems or finger stick techniques. Such analytes are then examined either using commonly available test equipment, examined at home with commercial test kits or materials or in an expensive clinical laboratory setting. While this is adequate for many applications, the increased control of certain analytes will require more real-time or extensive data for adequate analyte control. In the hospital environment, in particular, for critical care cases, improved survival rates and treatment costs can be obtained if real-time measurement in blood, urine or other bodily fluid, of calcium, blood oxygen levels, glucose, electrolytes such as potassium and calcium, and other physiological parameters related to critical care, can be measured in a real-time basis. The frequent and real-time assessment of these and other analytes can substantially improve clinical diagnosis and management, particularly in order to avoid great variations in the analyte concentration and to avoid hospitalization. This is particularly true with organic analytes containing hydroxyl groups (—OH) such as glucose.

Similarly, in ex vivo analysis, involving non-physiological hydroxyl-analytes such as commercial materials, such as ethylene glycol, glycerin, 1,4-butanediol, any soluble mono- di- or higher saccharide, can require real-time results of content to control costs and to improve productivity and product quality. Other commercial applications of the system or sensor include the analysis of a variety of commercial sugars, sweeteners or saccharides, including sugarcane juice; analysis of food adulteration using nonnutritive sweeteners; analysis of d-xylitol production output; analysis of fructose production output; analysis of a variety of nutritive and nonnutritive carbohydrates in foods; determination of the carbohydrate profile in fruit juice products to detect unwanted adulteration; analysis of sugar acids in wine and must; and other commercial applications where such real-time analysis can improve product quality and productivity.

In general, and in the in vivo analysis embodiment, the glucose, hexoses and other hydroxyl compounds, as related to diabetes, is a model for such a need for a useful system. In some cases, real-time monitoring of glucose in disease prophylaxis is essential. Type 1 diabetes, and to a lesser extent type 2 diabetes, is a chronic disease that can present large fluctuations in blood glucose levels. Glucose can range in healthy individuals from a fasting normal of about 70 to about 95 milligrams per 100 milliliters. In ill diabetic patients, the blood glucose level can drop substantially below 70 milligrams per 100 millimeters and can rise substantially above 100 milligrams per 100 milliliters indicating potentially severe medical problems. The goal of diabetes therapy is to maintain a glucose concentration that ranges from about 75 to about 95 milligrams per 100 milliliters without substantial deviations from the normal concentration.

Hypoglycemia, low blood sugar, if substantially below normal can result in coma and death. Hyperglycemia, in both type 1 and 2 diabetes, can also cause severe physiological damage leading to coronary artery disease, hypertension, problems with eyes, nerve damage, kidney damage and other problems. Prior art glucose clinical analysis methods and clinical and home glucose sensors have substantially relied on the chemical/biochemical species (e.g.) glucose oxidase in the colorimetric determination of glucose levels. Chemical methods, such as these, are based on a redox system involving oxidation reduction materials that use blood glucose as a reactant and through an oxidation reduction potential produce a chemical color change proportional to glucose concentration. Further, electro-chemical glucose sensors use immobilized bio-molecule enzyme compositions, such as glucose oxidase, deposited on metallic electrical sensors that measure glucose concentration using electrical signals obtained from oxidation reduction reactions that produce a free electron that can be measured in proportion to glucose concentration. While a variety of noninvasive methods have been tried, a substantial need exists for improved rapid or real-time measurement of hydroxyl compound, hexose or glucose concentrations, in patients generally and particularly in high risk diabetic patients.

Diabetes is one of the most significant global health challenges of the 21^{st} century. It remains one of the leading causes of death and is a major contributor to cardiovascular disease and is the leading cause of kidney failure, non-traumatic lower-limb amputation and new cases of blindness in the United States. Worldwide, the predominance and occurrence of diabetes has reached epidemic proportions and is expected to grow to 438 million by 2030. Currently, diabetes is not curable but can be controlled through proper management, which includes accurate monitoring of blood glucose levels, in order to improve lifestyle and lifespan. Effective and consistent monitoring, which is essential for accurate monitoring, remains a barrier to proper control of this disease due to the invasive and costly nature of currently available monitoring devices and resulting poor patient compliance.

Currently, the self-monitoring blood glucose test is the cornerstone of self-management for patients with diabetes. Unfortunately, this test requires that the patient extract a small drop of blood through an inconvenient and painful finger or torso pricking method three to four times daily for type I diabetes, according to the American Diabetes Association. In addition to this motivational barrier, high out-of-pocket expenditures for device test materials are also cited for non-compliant testing. Over time, suboptimal testing frequency leads to out-of-range blood glucose levels and potential health complications.

Positive societal and economic impact can be achieved with the development of an easy-to-use, implantable glucose monitoring system. An implantable device is beneficial to patients because it provides real-time continuous information regarding glucose levels. Early detection of rapidly changing glucose levels is especially important for patients with type I diabetes when the onset of hypoglycemia can come without warning and can incur potentially dangerous consequences. An implanted data signaling and sending device (i.e.) an RFID enabled device would minimize the continual cost, pain and complications of current diagnostic systems. In terms of limiting expense and increasing comfort and testing compliance, diabetic patients would benefit from the long operational life of a one-time invasive, implanted device. An implantable glucose monitoring device is superior to other systems because, although initially more invasive upon implantation, ultimately and for the long-term it is non-invasive on a daily basis. Ease of use makes patient monitoring and compliance a relative non-issue compared to the requirements of sampling blood daily or using an invasive, transdermal cannula. Additionally, glucose fluctuation data can be gathered electronically and stored for observation in real-time with no input from the patient. This embodiment gives an overview of the design and proof-of-concept development of a self-contained and closed-cycle, stable glucose sensing system as the integral component of an implantable device for real-time in vivo glucose measurement and diabetes management.

Since the advent of the first commercial glucose testing devices in the 1970s, there has been progress toward the development of glucose detection techniques designed for non-invasive systems. The three most studied techniques include enzyme, fluorescent and NIR spectroscopy. Despite various attempts, successful development of a fully functional implantable, non-invasive continuous monitoring device has remained elusive due to critical deficiencies of these detection techniques. Each method has physical and/or chemical limitations that make them impractical for use in a long-term, implantable device. Enzyme-based techniques function on reagents that are consumed and require a continuous reagent supply during the process of detection. The by-products of the reagent reactions are undesirable and cause detection interference. In addition, enzyme based detection techniques experience reagent degradation and inactivation over the long-term, eventually causing inaccurate readings and sensor drift. Similar to problems with enzyme-based techniques, there are also reagent limitations for long-term fluorescence-based systems. Current fluorescence based sensors cannot remain at an implantation site and respond to blood glucose concentrations over an extended period of time. Over the lifetime of the sensor, denaturation, relaxation, or poisoning of the fluorescent molecular recognition element occurs. Gradual deterioration of signaling reagents results in sensitivity and signal shifts that subsequently require continual readjustment and calibration in order to achieve accurate measurement. Using NIR spectroscopy to decipher glucose levels by way of absorption measurements through or at tissues, however conceptually simple, is equally impractical. This approach is currently not acceptable for clinical use due to the fact that a number of factors such as tissue hydration, blood flow, temperature, light scattering and overlapping absorption by non-glucose molecules cause read-out precision errors. It is no surprise that the search for the ideal glucose detection system continues to motivate the scientific community. However, past efforts in designing an implantable and self-contained glucose sensing system have not been successful because developers have given only partial consideration to the long-term impact and limitations of the in vivo environment.

### BRIEF DISCUSSIONS OF INVENTION

A technically and commercially successful implantable glucose sensor requires the integrated design and development of several critical components. (FIG. 9 shows a block diagram of one embodiment of a sensor.) The mission-critical self-contained and closed-cycle sensing component must be designed to interface with an appropriate signal transduction/signal processing device that, in turn, is coupled to the sensor's electronics and communication function. Further, the entire device must be enclosed in a porous, biostable and biocompatible material that simultaneously prevents biofouling of the device and allows biotransport of the glucose analyte in and out of the device. Failure to integrate any of these components into the implantable device invariably leads to product development failure. FIG. 10 shows one embodiment of the sensor that can be implanted in a location and read remotely. The sensor comprises a sensing system 20 and a communication system 20. In FIG. 10 the glucose 11 penetrates membrane 12 and forms concentration of glucose 11 in the sensor 10. The competitive binding environment 15 and glucose 11 compete for the competitive/signaling component 13. The ratio of binding between the competitive/signaling component 13 and glucose 11 cause mass-based signal detection 16, 17. An electrical signal from circuit 18, an application specific integrated circuit (ASIC) (mechanical or electrically generated sensor circuit), can send a signal from the antenna 19. This signal can be read remotely

One design for the implantable device, as illustrated in FIG. 10, envisions signal transduction using a MEMS cantilever that will respond to bound/unbound mass changes of the reporter construct with subsequent processing of the resulting signal on a device-specific ASIC chip. Signal export to the external environment will be via RFID communication with signal processing to provide the diabetic patient and their medical team with glucose concentration and rate-of-change information both on-board the RFID reader module and wirelessly exported to an external database. Additionally, the biocompatible/biotransport membrane will: 1) protect the device from encapsulation and 2) facilitate the size-selective transport of the low molecular weight fraction of the in vivo fluid matrix in and out of the device, while also containing the mobile sensing system reagents FIG. 10. While each of these component pieces is integral to the success of the device, the sensing system is the mission-critical component.

We have found a system for detecting or analyzing an analyte in in vivo, ex vivo or in vitro systems that can be adapted to rapid, real-time or continuous detection and analysis. The system uses a competitive mechanism such that an analyte competes with an immobilized polyol competitor surface for a dendrimer-boronic acid competitive/signaling component. This competition in a variety of embodiments can produce a useful measure of an analyte concentration. The dendrimer-boronic acid component can reversibly bind to the analyte and can also reversibly bind to the immobilized competitor surface. The degree to which the dendrimer component binds to either the analyte or the immobilized competitor surface can provide a measure of analyte concentration in a number of embodiments. Each binding association has an associated binding constant K_{eq} (K_{ad} or K_{id}, see FIG. 3). The K_{id} is the binding constant between the dendrimer-boronic acid and the immobilized polyol. The K_{ad} is the binding constant between the analyte and the dendrimer-boronic acid. Each component, the immobilized competitor surface and the dendrimer-boronic acid component, each with its associated binding constant, is chosen to provide the correct degree of competition such that the competitive binding is indicative of or is proportional to the concentration of the analyte. The binding component (to the analyte or polyol) of the dendrimer-boronic acid (DBA) of this system is the boronic acid on the dendrimer. In this example, the dendrimer-boronic acid component is the only component that reversibly binds to the surface. The analyte, within the scope of this example, does not bind to the surface, (i.e.) the binding constant between the analyte and the surface is substantially less than that of K_{ad} or K_{id}. The competitive interaction that we see is the analyte (glucose) competing with the immobilized diol for the boronic acid dendrimer-boronic acid component. As a result, a binding constant exists between each of the units (see FIG. 3) that competitively bind with the dendrimer-boronic acid component, the first being the binding constant between the dendrimer-boronic acid component and the analyte and the second being the binding constant between the dendrimer-boronic acid component and the immobilized diol surface.

We have designed and demonstrated a sensing system based on a dendrimer-boronic acid signaling component (DBA) and immobilized saccharide mimic (iDIOL). Our materials ultimately do not require a fluorescent dye molecule to signal glucose concentration through DBA:glucose:iDIOL competition, as the device will function through a mass-sensitive or mechanical, signal transduction interface. We have also found that the careful fractionation/selection of preferred molecular weights, surface functional groups, and functional group loading levels enhance competition and K_{eq} of the system. In addition, the system components were synthesized with favorable aqueous solubility and stability characteristics. Each component was designed to include optimal structural motifs for the most favorable glucose sensitivity and selectivity. Faced with the challenge of sensing a range of physiologically-relevant glucose concentrations in a complex matrix of potentially competing analytes, we developed a competitive binding model to expedite screening of our system components. Coordinated identification of DBA:iDIOL pairs that competitively interact with glucose was based on our evaluation of the K_{eq} between a DBA and an diol (as precursor to an iDIOL) versus the K_{eq} between the DBA and glucose.

Regardless of the detection system used to measure the release of the dendrimer-boronic acid component, the binding constants are such that the detection or analysis provides a useful result. We have found that the size or mass and the structure of the dendrimer-boronic acid component, or fraction thereof, provide convenient, precise and real-time detection and analysis. We have found that the detection system of the invention can be used to generate reproducible analyte concentration curves in physiologically relevant analyte concentrations

We have found that the detection system of the invention can be used in at least a fluorescent mode in which fluorescence can be used. In the analysis, we change the location of the fluorescent material within the overall system such that (1) it may or may not receive the excitation light causing only some proportion of the total label present to fluoresce and/or (2) the fluorescence sensor only “sees” the fluorescent label that is on the immobilized surface or in free solution.

We have also found that the detection system can be used in a micro-cantilever detector. In the micro-cantilever detector, the mass of the dendrimer component as it is bound to or displaced from the cantilever, changes the mass on the cantilever and provides a detectable and useful signal.

We have also found that the detection system of the invention can be used in a mammalian or human sensor that can be used at or on the skin surface or subcutaneously to give rapid, continuous and real-time information. In the subcutaneous sensor, we have designed a chemical system in which the components within the system can competitively interact with an analyte that penetrates the sensor structure so as to respond either directly or inversely proportional to the physiologically relevant blood analyte levels providing a meaningful detection or quantification response. We have found that a substantial and useful subcutaneous glucose sensor can be manufactured in a unit comprising the sensor in a container sealed with a selective membrane to select the analyte or with a molecular weight cut-off membrane, to retain the dendrimer in the sensor, if needed, and to help reduce or prevent unwanted interference with the analyte. Within the container is placed a sensor that detects or quantifies the analyte. In one embodiment, the sensor can use a fluorescent mechanism to detect or quantify the analyte. In the second embodiment, the detector can use a piezoelectric micro-cantilever sensor that provides a stable electrical frequency output as the analyte displaces the comparatively (with respect to the analyte) massive dendrimer structure from the cantilever.

We have found that the binding constant (K_{ad}) between an analyte such as glucose (K_{gd}) and similar constant (K_{ad}) between an analyte and dendrimer, preferably a dendrimer-boronic acid component, or fraction thereof, can be used within a range of ratios and coordinated with the constant K_{id} of the dendrimer-boronic acid to the polyol immobilized on a surface of a detector structure. The competition between analyte (glucose) and the diol immobilized on the surface for the dendrimer-boronic acid component provides the signal used in detection or quantification. We have found that the system of the invention used either in a fluorescent mode or in a piezoelectric micro-cantilever mode can generate reproducible glucose concentration curves in physiologically relevant glucose ranges (30-1000 mg per 100 mL of serum or plasma).

Increasing demand for the detection of bioanalytes has triggered the development of rapid assay techniques in the form of sensor technologies. The need for more robust sensors that transcend the cost and stability limitations of current detection systems that require consumable biochemical reagents, such as enzymes and antibodies, has fueled the trend toward the design and development of sensing systems that are based on synthetic components like aptamers, MIPs and receptor constructs. In addition, detection of bioanalytes may require more advanced sensing component materials in order to substantially increase sensitivity and selectivity due to the complexity of the sample matrix and the inherently low analyte concentration that can exist in a physiological system. The approach of utilizing synthetic materials for the construction of chemical recognition systems provides the structural and functional materials required for effective and robust sensing/receptor function. Developing synthetic recognition materials with known physical and chemical properties provides the advantage of flexibility in selecting compatible sensing system reagents that meet the design criteria for operation within a physiological environment. It is critical that the reagents simultaneously function in complex, aqueous media while maintaining performance integrity under physiological pH and temperature. It is also imperative that the materials not only preserve sensitivity and selectivity within complicated matrices of potentially competing analytes, but also retain sensitivity for a particular moiety whose physiological concentration may be low. The design challenges of a in vivo sensing system can be overcome using synthetically optimized recognition materials.

The applicability of artificial receptor materials to the development of hydroxyl compound, hexose or saccharide sensors, especially as it relates to glucose detection, has attracted a great deal of interest. Efforts to improve signaling technology continue to make headway because materials with enhanced biocompatibility and superior sensitivity and selectivity toward glucose are fundamental requirements for monitoring such hexose or glucose levels in an implantable device. Our group has developed a sensing system technology for in vivo glucose analysis that utilizes synthetically optimized materials to fulfill the reagent requirements of a self-contained and closed-cycle, stable glucose sensing system. We have successfully developed components that can detect biologically relevant levels of glucose with the required sensitivity and selectivity in a physiologically relevant matrix solution. The materials are physically and chemically stable in aqueous media at physiological pH. Scalability is also an advantage of these reagents, in that they are reproducible on a large scale with the capability to meet commercial demand.

The novel approach to glucose sensor design devised by our group involves two main components: a synthetically optimized boronic acid terminated dendrimer scaffold and a surface immobilized monosaccharide mimic. When these components are exposed to glucose, they competitively interact to produce a detectable and reproducible signal that is responsive to fluctuating levels of glucose. The magnitude of sensitivity and selectivity is tunable through the use of appropriate boronic acid and dihydroxy (polyol) analogues (iDIOLs) and the degree of sensitivity and selectivity can be optimized based on a system specific binding affinity model and database. Reported herein is an overview of the development of our synthetic glucose sensing system. This description includes a discussion of our strategy, along with an overview of the in-depth considerations we used to select system components for optimal detection performance in a physiologically relevant environment.

### BRIEF DESCRIPTION OF FIGURES

FIGS. 1-3 show the mechanism of competition between the analyte and the dendrimer structure on the polyol of the immobilized surface.

FIGS. 4-5 are graphical representations of the analysis results of glucose using the system of the invention.

FIGS. 6-8 show the structures of selected, polyols, dendrimer, boronic acid and dendrimer-boronic acid component materials of the invention.

FIG. 9 shows one embodiment of a bioselective interface between the in vivo environment and the sensing system, the closed-cycle glucose sensing system and a mass-sensitive signal transduction interface that is coupled to the RFID-enabled data communication component.

FIG. 10 shows an embodiment of a glucose sensor that will integrate the glucose sensing system with a mass-sensitive signal transduction mechanism coupled to the RFID-enabled communication electronics, all enclosed in a millimeter scale, implantable package. Any communication system will work and any electrical or mechanical signal transduction system will work.

FIG. 11 shows glucose competition curves showing normalized fluorescence intensity versus glucose concentration for boronic acid 1 and boronic acid 2 (See Table 1) in a physiological buffer at neutral pH.

FIG. 12 shows diol competition curves showing normalized fluorescence intensity versus diol 1 and diol2 (See FIG. 13) concentration for a DBA (See Table 2) in a physiological buffer at neutral pH.

FIG. 13 shows structures of DBA 2 (A) (See Table 2), diol 1 (B) and diol 2 (C) evaluated for binding performance in a diol competition binding assay.

FIG. 14 shows glucose competition curves showing the normalized DBA fluorescence intensity versus glucose concentration for DBA 1, DBA 2, and DBA 3 in physiological buffer at neutral pH on an iDIOL 3 surface.

FIG. 15 shows structures of DBA 1 (A), DBA 2 (B), DBA 3 (C) and iDIOL 3 (D) evaluated for binding performance in a glucose competition binding assay.

FIG. 16 shows IC₅₀ values from glucose competition response curves of various DBA:iDIOL combinations.

FIG. 17 shows glucose competition curves showing the normalized DBA fluorescence intensity versus glucose concentration of DBA 3 in a physiological buffer at neutral pH on an iDIOL 1, iDIOL 2 and iDIOL 3 surface. Binding constants, in the K_{eq} interaction graph, for DBA 3:glucose and DBA 3:diol 1, 2, and/or 3 as precursors to iDIOL 1, 2, and/or 3 combinations were correlated with the glucose response curves of each DBA:iDIOL system.

FIG. 18 shows chemical structures of iDIOL 1 (A), iDIOL 2 (B), iDIOL 3 (C) and DBA 3 (D) evaluated for binding performance in a glucose competition binding assay.

FIG. 19 shows glucose, fructose and galactose competition curves showing the normalized fluorescence intensity of DBA 3 versus saccharide concentration in a physiological buffer at neutral pH on an iDIOL 3 surface.

FIG. 20 has data about binding constants for selecting useful pairs.

### DEFINITIONS

ARS Alizarin Red S; also known as 3,4-Dihydroxy-9,10-dioxo-2-anthracenesulfonic acid sodium salt
IC₅₀ Half Maximal Inhibitory Concentration
Keq Equilibrium constant
MIP Moleculary imprinted polymer
NIR Near-infrared
PET Photoinduced electron transfer
pKa The negative logarithm of the dissociation constant
RFID Radio frequency identification

### DEFINITIONS

DBA Dendrimer-boronic acid—A dendrimer construct that is functionalized with boronic acid receptor ligands and a fluorescent reporter moiety. The DBA is the sensing system signaling component that can competitively bind to the glucose analyte and to the 1,2- and 1,3-dihydroxy motif(s) of iDIOLs.
diol A saccharide analogue moiety that typically contains a 1,2- or 1,3-dihydroxy motif and a functional group that can be used for covalent immobilization of the iDIOL on a support to create an iDIOL.
iDIOL Immobilized diol/saccharide analogue—An immobilized diol/saccharide analogue that contains a 1,2- or 1,3-dihydroxy moiety that is covalently attached to a support. The iDIOL competes with glucose for DBA binding, which produces a bound versus free sensing system signal.
DBA:glucose:iDIOL Designation of the three component competitive system where glucose and the iDIOL compete for DBA binding to produce a signal response that is proportional to glucose concentration.

For the purpose of this disclosure, the term K_{id} refers to the binding constant between a dendrimer-boronic acid component and an immobilized polyol on the surface.

For the purpose of this disclosure, K_{ad} refers to the binding constant between an analyte and a dendrimer-boronic acid.

For the purpose of this disclosure, K_{gd} refers to the binding constant between an analyte such as glucose and a dendrimer-boronic acid.

For the purpose of this disclosure the term immobilizes/immobilized means that a compound is bonded to a surface with bond strength similar to a covalent bond and that bond strength is greater than a reversible bond keeping the immobilized compound on the surface during the competitive reactions of the analyte and the dendrimer-boronic acid component with the immobilized polyol.

For the purpose of this disclosure the term reversible bond or reversibly bonded indicates bond strength less than a covalent bond and a bond that can be disrupted by competition with a compound with a generic constant (i. e.) K_{eq} similar in strength (i.e.) within about an order of magnitude.

For the purpose of this disclosure the term compete means that the K_{eq} of two competing molecules to a binding site are close enough in value that a first molecule can displace a proportion of the other molecule at equilibrium.

For the purpose of this disclosure the term DBA refers to a dendrimer-boronic acid component.

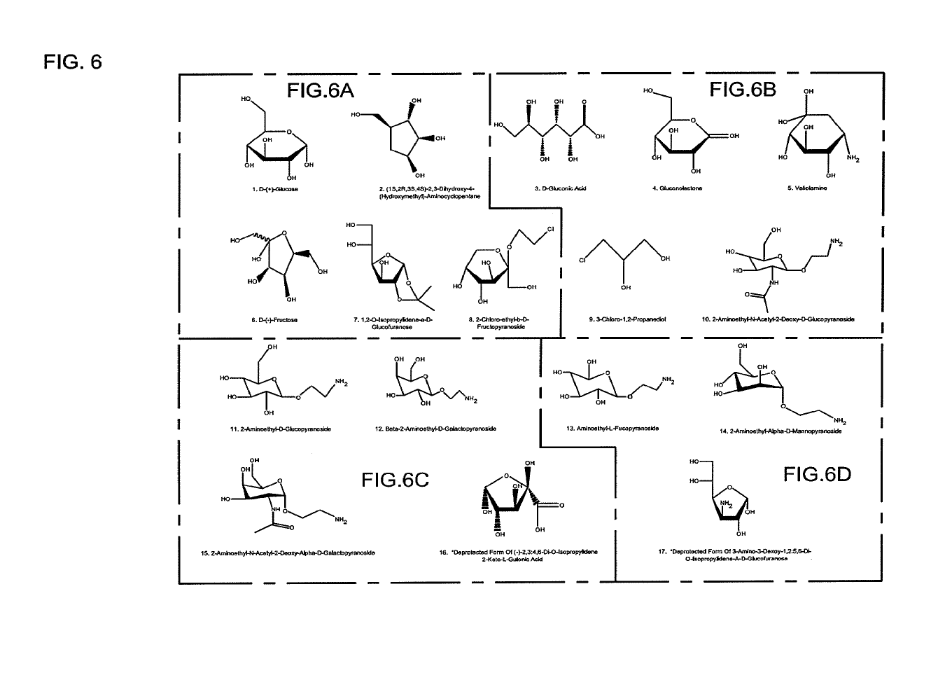

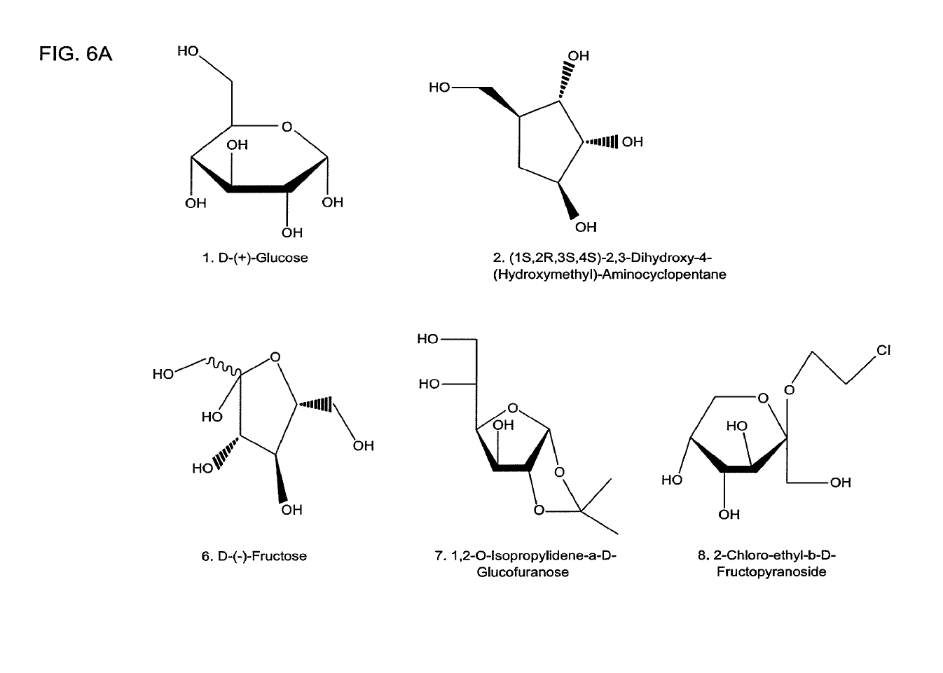

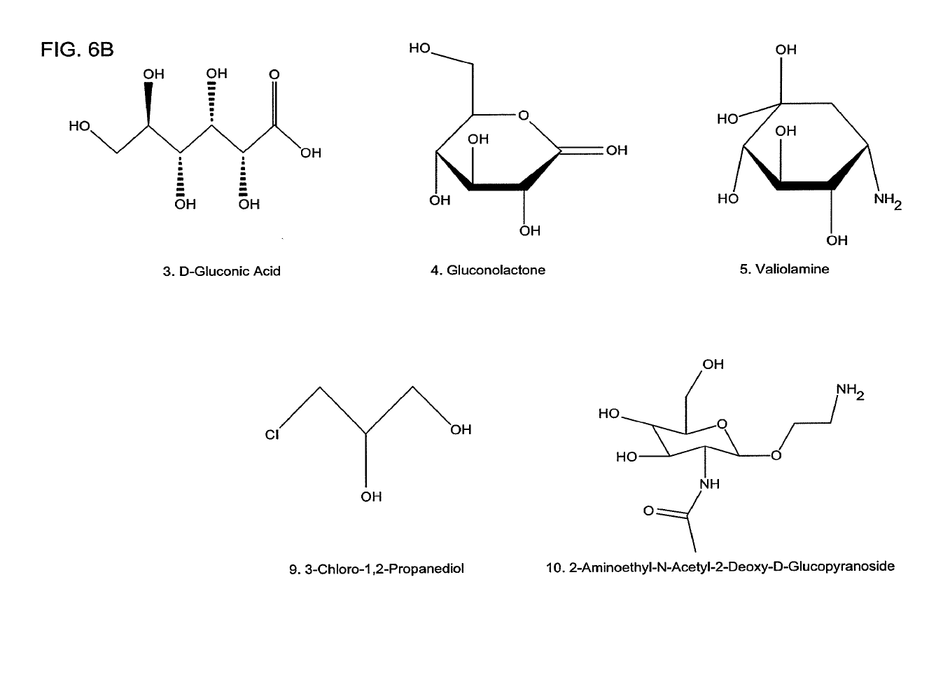

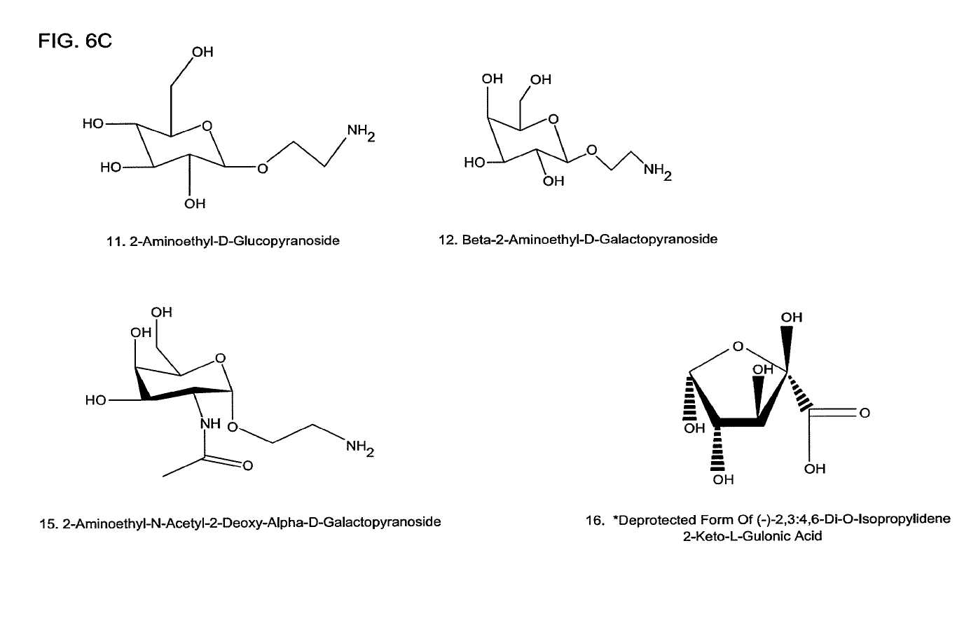

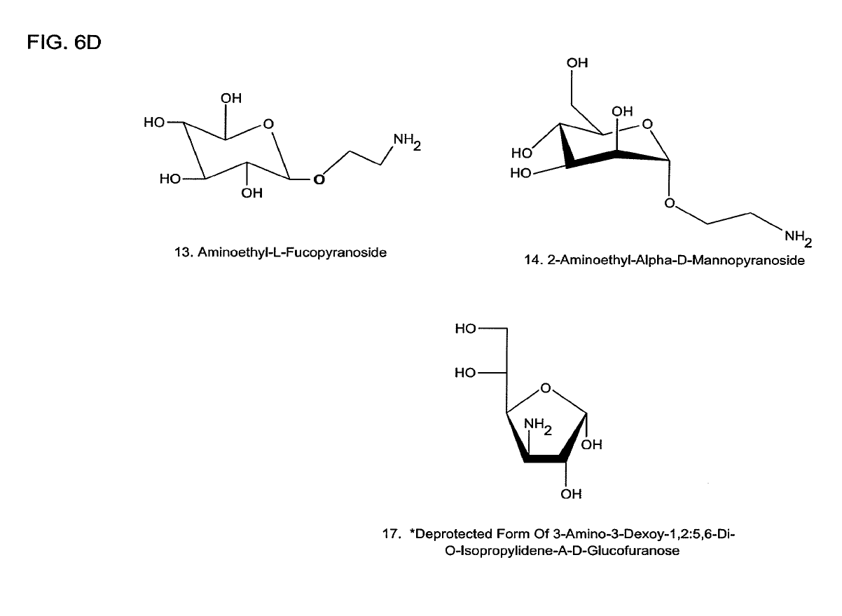

For the purpose of this disclosure, the term polyol refers to an organic compound with at least two hydroxyl groups, including alkylene polyols and natural and synthetic carbohydrates and derivatives thereof. The term polyol means a compound that contains at a minimum the structure:

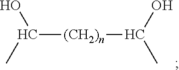

wherein n=0-5 and the carbons are aryl or aliphatic and the empty valences indicate additional structure or covalent attachment to the surface. The polyol is a generally hydrophilic compound. As polyol compounds, there may be mentioned hydrophilic polyols that include glycerin, poly(vinyl alcohol), poly(ethylene glycol), polypropylene glycol), etc. Other polyols include oligo-, di- and mono-saccharides such as sucrose, mannitol, lactose, L-arabinose, D-erythrose, D-ribose, D-xylose, D-marmose, D-galactose, lactulose, cellobiose, etc. Preferred polyols are a natural or synthetic saccharide compound or a saccharide mimic. FIG. 6 shows an array of useful polyols that can be immobilized to the surface in the system of the invention. The —OH group of the polyol must be available on the surface to reversibly bind to the DBA and compete with the analyte.

### DETAILED DISCUSSION

In the detection or quantification system of the invention we have found that a polyol immobilized on a surface can be used in a competitive system. Bonding between the polyol and the dendrimer-boronic acid has a binding constant K_{id}. Bonding between the analyte and the dendrimer-boronic acid has a constant K_{ad}. The analyte and polyol compete to bind to the dendrimer-boronic acid proportionally to the concentration of the analyte. At a constant concentration of analyte, as the system reaches equilibrium such that a proportion of the dendrimer-boronic acid component is bonded to the surface and the balance is bonded to the analyte.

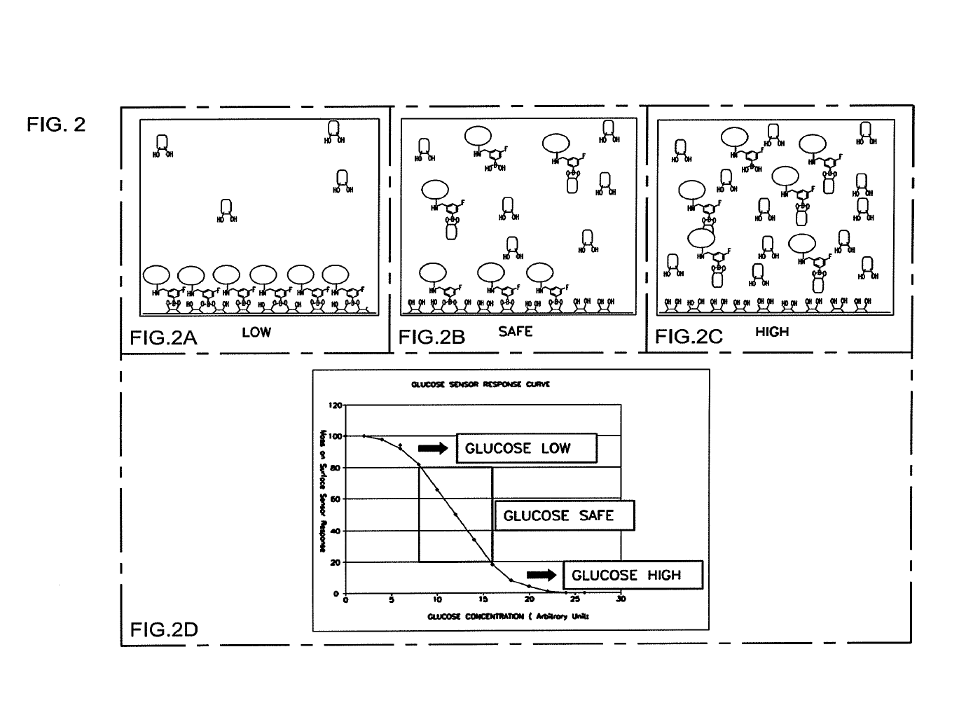

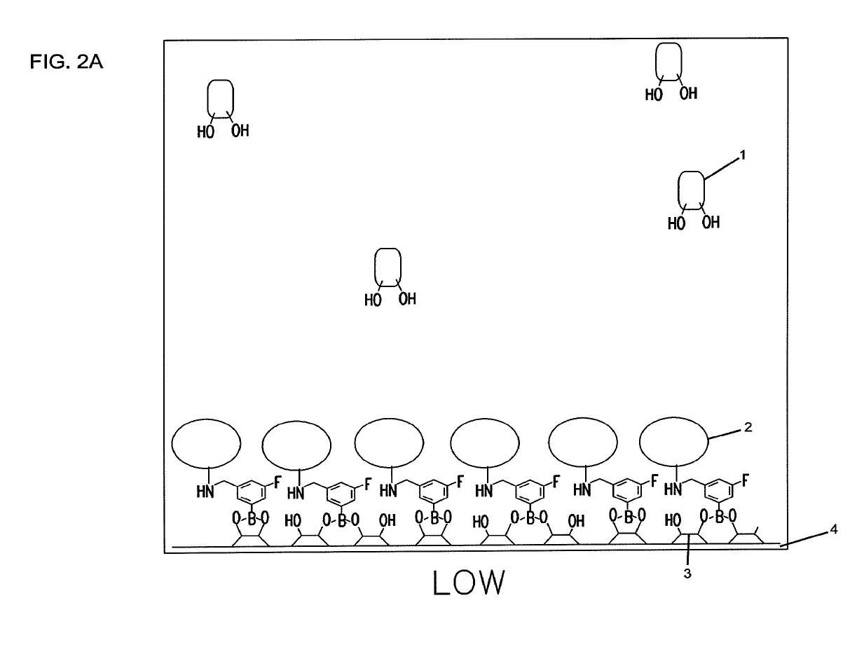

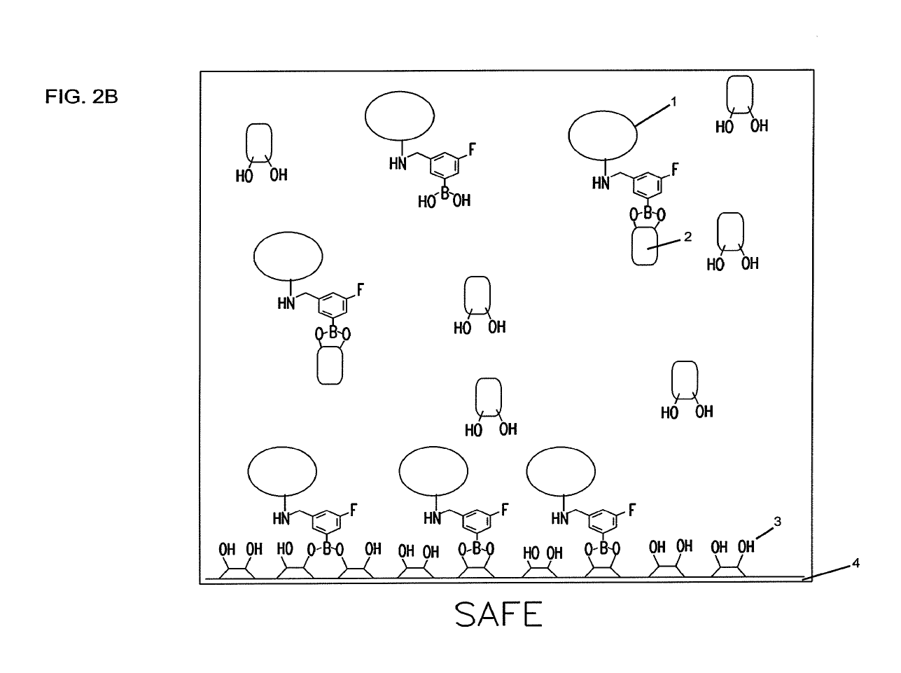

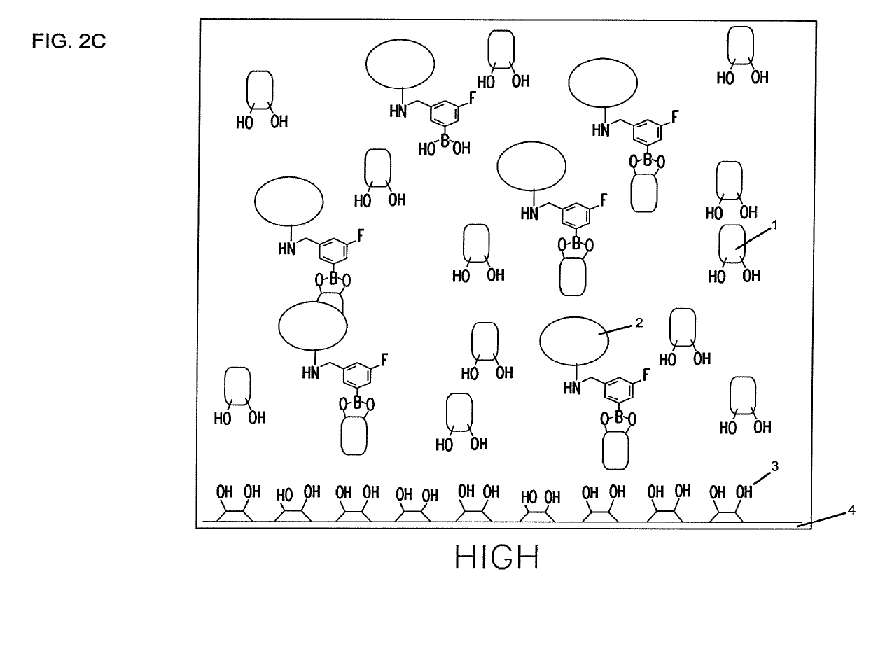

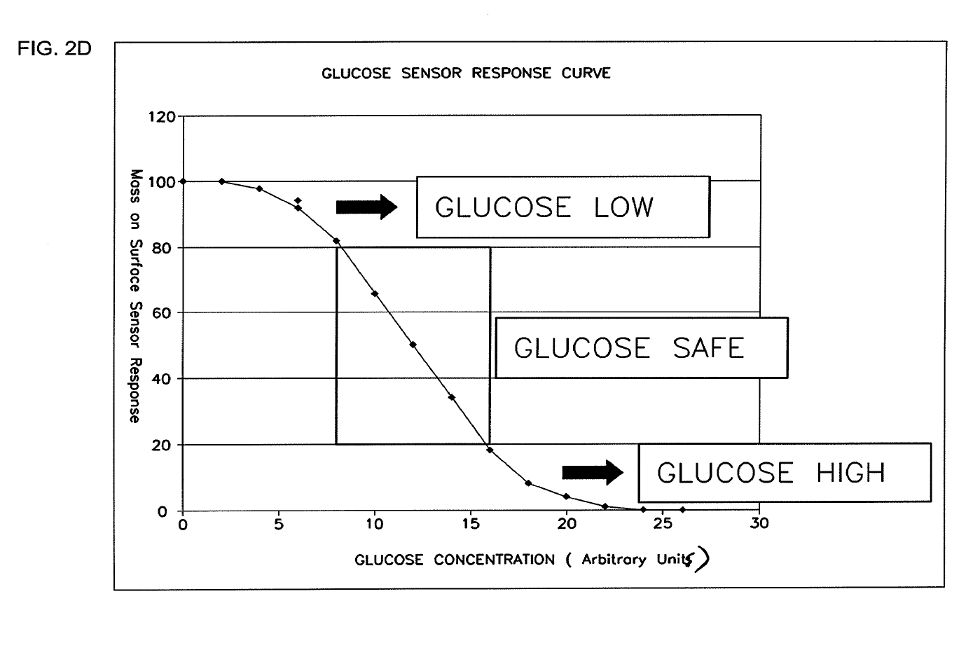

The design of our device is based on the creation of an integrated, self-contained sensing system that produces an RFID read-out, which provides two pieces of information: milligrams per deciliter (mg/dL) glucose values and an indication of whether the physiological glucose concentration is increasing or decreasing. This combination of information can be used by the diabetic patient to determine whether their glucose levels are currently low, safe, or high (FIG. 2D). Demonstration of the closed-cycle chemical sensing system required the interaction of two components. These components are: 1) the competitive agent/signaling component, which is based on a dendrimer-boronic acid (DBA) construct (FIGS. 2) and 2) the glucose-competitive DBA binding environment, which consists of an immobilized monosaccharide mimic (iDIOL, FIG. 2). Our unique detection approach functions through reversible competitive binding between glucose and the iDIOL for the DBA. The amount of DBA that is bound to the iDIOL binding environment on the mass-sensitive transduction interface fluctuates in response to changing levels of glucose. The change in free versus bound DBA is measured via a change in the resonance frequency of the MEMS microcantilever. This signal transduction event gives a measurement of glucose concentration that can be calibrated to bloodstream glucose levels (FIG. 2). The function of this type of sensor relies on the relative affinity of glucose and the iDIOL for the DBA. Consequently, optimization of the glucose sensing system was based on our evaluation of the binding affinities of the DBA for both glucose and the iDIOL. More broadly, our approach for constructing and optimizing component materials was also based on an in-depth consideration of how these materials related to the sensing system and the device as a whole.

### DETAILED DISCUSSION OF FIGURES

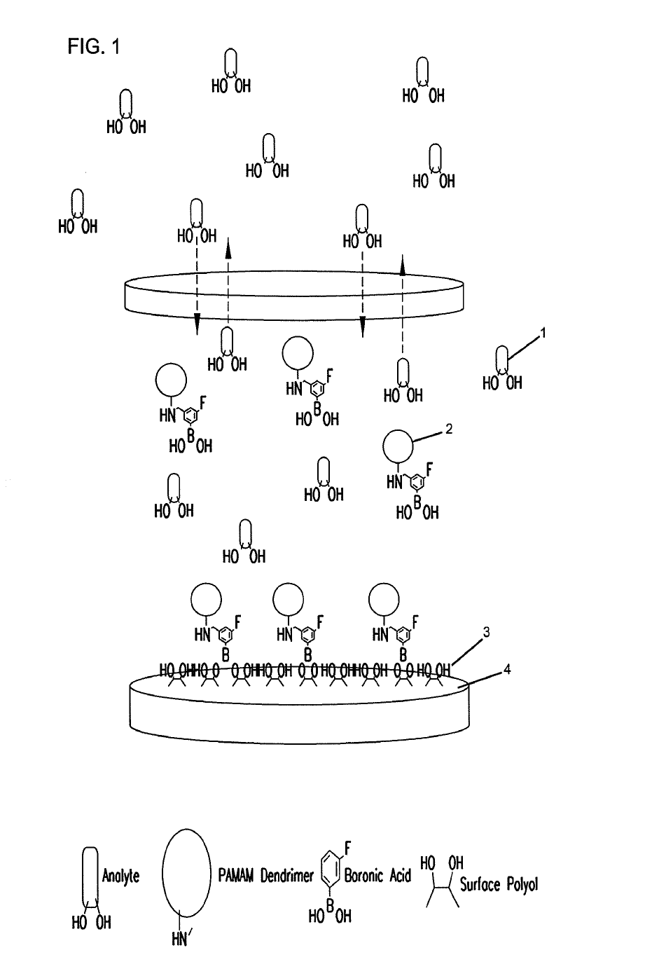

FIG. 1 is a graphical representation of the competitive interaction of the analyte 1 and of the dendrimer-boronic acid component 2 for the immobilized polyol 3 on the surface 4. We have found that the competitive nature of select dendrimer-boronic acid components for an immobilized polyol on the surface can be utilized in the system of the invention.

FIG. 2 is a more detailed graphical representation of the competitive interaction of the analyte 1 and the dendrimer-boronic acid component 2 for the immobilized polyol 3 on the surface 4 at varied concentrations of analyte. As can be seen, for low concentrations of analyte 1, few if any dendrimer-boronic acid components bind to analyte and are not displaced from the immobilized polyol. In physiologically normal analyte concentrations, some dendrimer-boronic acid components are displaced from the immobilized polyol and are bound by or to the analyte. At high concentrations of analyte, substantial numbers of dendrimer-boronic acid components, if not all, are displaced from the immobilized polyol surface. In the graph of FIG. 2, the proportional response to the mass change on the surface immobilized polyol can be graphed against analyte (glucose) concentration, providing useful information.

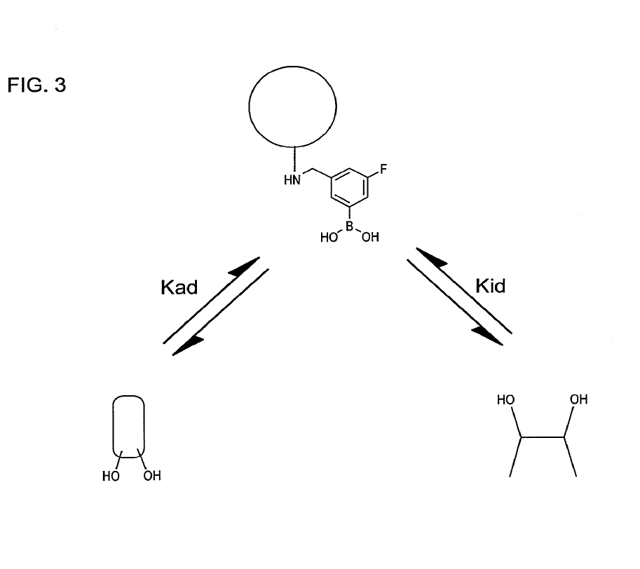

FIG. 3 is a graphical representation of the competitive structure between a analyte 1 and an immobilized polyol 2 for the dendrimer-boronic acid component 3. As discussed above, the binding constant K_{ad} quantifies the bond strength between the analyte and the dendrimer-boronic acid component/construct/boronic acid. The binding constant between the analyte and the dendrimer-boronic acid component and the binding constant between the dendrimer-boronic acid component and the immobilized polyol surface must be balanced and kept within useful proportions. There is a K_{ad} between the dendrimer-boronic acid components and the analyte. There is a K_{id} between the dendrimer-boronic acid components and the immobilized polyol. Typically the ratio of K_{id}:K_{ad}=about 0.1 to 10 or about 0.5 to 2. Within these ratios, a detectable amount of dendrimer-boronic acid component competes with the analyte and the immobilized polyol surface to produce a useful signal. As this ratio substantially departs from these ranges the dendrimer-boronic acid component will tend to bind to either the analyte or the immobilized polyol on the surface and will not give a useful analytical response or determination.

### Dendrimers DBA

In a preferred mode of practicing the invention, we have found that the DBA can be easily synthesized with reproducible results. We have found that the dendrimer-boronic acid scaffold or structure is stable and has a K_{ad} and K_{id} that can be used in analyte analysis or detection generally and can also be used in glucose analysis and detection.

Macromolecular DBA constructs have been used for the first time by our group as the glucose recognition and signaling agent in a competitive binding assay that will ultimately be incorporated as a mass-sensitive detection method for the in vivo determination of glucose concentration. A dendrimer-boronic acid construct has been described for use in an in vitro saccharide sensor by James, et al. In this example, anthracene units are used as the dye indicator that correlates fluorescence intensity changes with saccharide binding. Although useful for detection of saccharides in an in vitro environment, this type of detection technique is not applicable to an implantable device for multiple reasons. The dendrimer constructs have limited aqueous solubility due to the highly insoluble anthracene moiety. More generally, the use of anthracene as a candidate for in vivo applications is unfavorable due to sensitivity issues, toxicity concerns and lack of metabolic stability. The viability of this type of sensor in a physiological matrix would be compromised, as the material would continue to loose sensitivity over time due to diminishing fluorescence resulting from denaturation, photodegradation and/or indicator poisoning. This would, in turn, require that the device be continually calibrated and frequently recharged with fresh reagents. In addition, there is not a well-established method for exciting the fluorophore and taking measurements from an implanted fluorescence-based device without inserting an invasive probe into the subcutaneous tissue. The design of our DBA constructs remedies these obstacles to functional implantation.

The first critical step required for demonstration of the glucose sensing system is the construction of the DBA competitive agent/signaling component. The selection of materials for the DBA component was dictated by the need to build synthetic receptor moieties that would respond with optimal binding sensitivity and selectivity for glucose in a complex aqueous matrix of potentially competing analytes. In addition, as deemed essential for extended function in a closed-cycle, long-term implantable device that is continuously exposed to the lytic nature of physiological fluid, the synthetic materials used to synthesize the DBAs must be stable and able to perform without diminished capacity over the lifetime of the sensor. Separately, but equally important, the materials must not be consumed during the detection process or require external reagents. For these reasons, our work focused on the development of a synthetic saccharide sensor that has the capacity to selectively detect glucose with long-term integrity in a physiological system.

Dendrimer and hyperbranched polymers are generally known. Dendrimers have a regularly repeated branching structure, while the hyperbranched polymer has an irregularly repeated branching structure. These polymers may have a structure in which the polymer chains are dendritically branched from one focal point, or a structure in which polymer chains are radiated from a plurality of focal points linked to a polyfunctional molecule serving as a core. Although other definitions of these species may also be acceptable, in any case, the dendritic component invention encompasses dendritic polymers having a regularly repeated branching structure and those having an irregularly repeated branching structure, wherein these two types of dendritic polymers may have a dendritically branching structure or a radially branching structure.

According to a generally accepted definition, when a dendritic structural unit extends from its preceding dendritic structural unit as a substantially exact copy thereof, the extension of the unit is referred to as the subsequent “generation.” The definition of a “dendritic polymer” according to the present invention covers those having a structure in which each of the dendritic structural units which are similar to one another with the same basic structure are repeated at least once also fall within the scope of the present invention.

The concepts in relation to dendritic polymer, dendrimer, hyperbranched polymer, etc. are described in, for example, Masaaki KAKIMOTO, Chemistry, Vol. 50, p. 608 (1995) and Kobunshi (High Polymers, Japan), Vol. 47, p. 804 (1998), and these publications can be referred to and are incorporated herein by reference.

In the dendritic polymer of the present invention, a dendritic structural unit is formed of a linear portion and a branch portion. The structure in which the dendritic structural unit is repeated once to provide a two-stage structure is in fact “a structure in which each of the branch portions of that structural unit is bonded to another with substantially identical structural units.” The resultant structure is referred to as a “1st-generation (1-G) dendron.” A similar structure in which dendritic units having the same structure are successively linked to the bonding end groups of the branch portions Y of a 1st-generation dendron is referred to as a “2nd-generation (2-G) dendron”. In a similar manner, an nth-generation (n-G) dendron is created. Such dendrons per se and dendrons to which a desired substituent or substituents are bonded to the ends or the focal point thereof are referred to as “dendrimers or hyperbranched polymers of dendritically branching structure.” When a plurality of dendritically branched dendrimers or hyperbranched polymers, which are identical to or different from one another, are bonded as subunits to a multivalent core, the formed dendritic polymer is called “dendrimer or hyperbranched polymer of radially branching structure.” Notably, a dendritic polymer in which nth-generation dendrons are linked to an r-valent core is defined as an nth-generation, r-branched dendrimer. Herein, a 1st-generation, 1-branched polymer in which the 1st-generation dendron is bonded to the mono-valent core also falls within the scope of the dendritic polymer of the present invention. In order to attain the objects of the present invention, dendritic polymers of greater than at least 1st-generation, 2-branched species or of at least 2nd-generation, 1-branched species are preferred. Generally, such dendritic polymers preferably have a molecular weight of 600 or more.

Preferred dendrimers and dendrons are substantially monodisperse and are usually symmetric, spherical compounds with surface reactive spherical groups. The field of dendritic molecules can be roughly divided into low-molecular weight and high-molecular weight species. The first category includes dendrimers and dendrons, and the latter includes dendronized polymers, hyperbranched polymers, and the polymer brush. In the system of the invention as molecular weight increases the precision and sensitivity of the analysis also tends to increase. At some point molecular weight can reach a level that degrades performance chiefly due to kinetic effects. The chemical or reactive properties of dendrimers are dominated by the functional groups on the molecular surface. In the dendrimer-boronic acid component, the dendrimer is selected such that the end of the branch (i.e.) on the molecular surface can couple and form a covalent bond to the boronic acid moiety; leaving the boronic acid group or groups formed on the surface free to bind with the analyte or immobilized polyol.

Dendrimers are also classified by generation, which refers to the number of repeated branching cycles that are performed during its synthesis. For example if a dendrimer is made by convergent synthesis (see below), and the branching reactions are performed onto the core molecule three times, the resulting dendrimer is considered a third generation dendrimer. Each successive generation results in a dendrimer roughly twice the molecular weight of the previous generation. Higher generation dendrimers also have more exposed functional groups on the surface, which can later be used to customize the dendrimer for a given application.

Poly(amidoamine), or PAMAM, is perhaps the most well known dendrimer. The core of PAMAM is a diamine (commonly ethylenediamine), which is reacted with methyl acrylate, and then another ethylenediamine to make the generation-0 (G-0) PAMAM. Successive reactions create higher generations, which tend to have different properties. Lower generations can be thought of as flexible molecules with no appreciable inner regions, while medium sized generation-3 or generation-4 (G-3 or G-4) dendrimers do have internal space that is essentially separated from the outer shell of the dendrimer. Very large (G-7 and greater) dendrimers can be thought of more like solid particles with very dense surfaces due to the structure of their outer shell. The functional group on the surface of PAMAM dendrimers is ideal for many potential applications. These dendrimer structures have a surface amino group that can react with a reactive group on the boronic acid to form the dendrimer-boronic acid component. A reaction between the dendrimer amino group and a formyl (—CHO) group on the boronic acid is one facile reaction mode.

Dendrimers can be considered to have three major portions: a core, an inner shell, and an outer shell. Ideally, a dendrimer can be synthesized to have different functionality in each of these portions to control properties such as solubility, thermal stability, and attachment of compounds for particular applications. Synthetic processes can also precisely control the size and number of branches on the dendrimer. There are two defined methods of dendrimer synthesis, divergent synthesis and convergent synthesis. However, because the actual reactions consist of many steps needed to protect the active site, it is difficult to synthesize dendrimers using either method. This makes dendrimers hard to make and very expensive to purchase. At this time, there are only a few companies that sell dendrimers; Polymer Factory Sweden AB commercializes biocompatible bis-MPA dendrimers. Dendritic Nanotechnologies Inc., from Mount Pleasant, Mich., USA produces PAMAM dendrimers and other proprietary dendrimers.

The dendrimer is assembled from a multifunctional core, which is extended outward by a series of reactions, commonly a Michael reaction. Each step of the reaction must be driven to full completion to prevent mistakes in the dendrimer, which can cause trailing generations (some branches are shorter than the others). Such impurities can impact the functionality and symmetry of the dendrimer, but are extremely difficult to purify out because the relative size difference between perfect and imperfect dendrimers is very small.

Dendrimers are built from small molecules that end up at the surface of the sphere, and reactions proceed inward building inward and are eventually attached to a core. This method makes it much easier to remove impurities and shorter branches along the way, so that the final dendrimer is more monodisperse. However dendrimers made this way are not as large as those made by divergent methods because crowding due to steric effects along the core is limiting.

### Boronic Acids

After synthesis of a dendrimer-boronic acid, the synthetic product can be fractionated to obtain fractions that vary in molecular weight, molecule diameter and the number of surface functional groups. Certain fractions have an optimized K_{eg} property. We have found that the use of appropriately designed boronic acids as molecular recognition units provides the ability to both selectively recognize and signal analytes, such as hydroxyl compounds, hexoses or glucose, at low concentrations and in real-time.

Numerous advances have been made in understanding how the electronic, geometric and polar properties of functional groups on boronic acid analogues affect the mechanism and process of reversible diol complexation. Several groups have demonstrated that saccharide selectivity and binding properties are affected by the location and type of substituents about the aromatic boronic acid substructure. It has also been reported that, in general, aryl boronic acids with lower pKₐ's tend to have higher binding affinities for diols near neutral pH, although optimal binding depends not only on the pKₐ of the boronic acid but also on the structure and properties of the diol in question, as well as the pH and ionic strength of the binding environment. Boronic acid pKₐ's are tunable by altering the substituents. For example, Badugu et al. (2005) have shown that the pKₐ of phenylboronic acid can be decreased by adding electron withdrawing groups, while adding electron donating groups increases the pKₐ. Alternatively, there is evidence that a neighboring nitrogen can enhance the formation of boronate esters under neutral pH conditions by coordinating intramolecularly with boron to create a more electron deficient atomic center, resulting in a reduction in the apparent pKa of the boronic acid. In our efforts to design a boronic acid-based receptor and signaling component, we exploited the physical and chemical influence of substituent type and location to improve the binding affinity and selectivity of DBAs for glucose and iDIOLs.

The ability of boronic acid-based sensors to function efficiently in a physiological system is reflected by their selective interaction with saccharides. For saccharide recognition to proceed, cyclic boronate ester formation must occur upon binding of a boronic acid to, preferably, a 1,2- or 1,3-diol to form a five- or six-membered cyclic ester. It is possible for boronate esters to form under aqueous conditions, but at neutral pH binding affinity is low. Greater binding affinity can be obtained under elevated pH conditions (pH 10), where the more favorable tetrahedral boronate form dominates. Designing a boronic acid-based sensor component that has greater binding affinity in a neutral physiological system can be achieved by: 1) strategically outfitting the phenylboronic acid substructure with electron withdrawing groups in the meta- or para-position in order to stabilize the boronate form of the acid and lower the pKₐ value and/or 2) introduce an ortho-amino methyl substituent to facilitate boronate ester formation at neutral pH through donation of the nitrogen lone pairs into the empty boron p-orbital. Strategic selection of boronic acid receptor molecules containing substituent(s) that have the greatest potential to initiate boronate ester formation was key in designing a signaling component that would perform with the desired glucose binding characteristics.

Our preliminary efforts in designing a boronic acid sensing system focused on selecting commercially available boronic acids with a diversity of substituent(s) about the phenylboronic acid substructure and a reactive group that could be used for coupling the boronic acid to a carrier scaffold. We selected phenylboronic acid molecules whose substituent type(s) and location(s) would increase the electrophilicity of the boronic acid group, reducing its pKa and ultimately, increasing the binding affinity at neutral pH. The resulting boronic acid dendrimer constructs, each of which possessed unique functionalities and enabled a diversity of saccharide binding sensitivities and selectivities, formed the basis of our library of candidate DBA signaling component materials.

One component of the dendrimer-boronic acid component is an organo-boronic acid, which can be represented as compounds I or II:

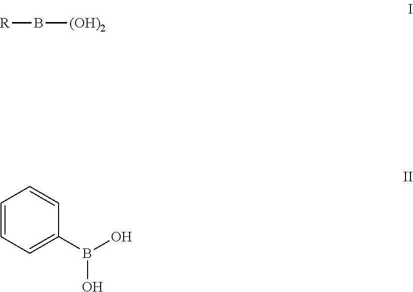

Boronic acids are conventionally made by the reaction of tri-alkyl borate and an aryl or unsaturated compound. Alternatively an alkene can be subject to the hydroboration reaction. In compound I, R— is an alkyl or aryl substituent on the boronic acid containing a carbon-boron bond belonging to the larger class of organo-borane compounds. Boronic acids act as Lewis acids. Their unique feature is that they are capable of forming reversible covalent complexes with sugars, amino acids, hydroxamic acids, etc. (molecules with vicinal, (1,2) or occasionally (1,3) substituted Lewis base donors (alcohol, amine, carboxylate)). The pKₐ, of a boronic acid is ˜9, but upon complexation in aqueous solutions, they form tetrahedral boronate complexes with pKₐ ˜7. They are occasionally used in the area of molecular recognition to bind to saccharide compounds for fluorescent detection or selective transport of saccharide materials across membranes. Boronic acids are used extensively in organic chemistry as chemical building blocks and intermediates predominantly in the Suzuki coupling. The trans-metallation of its organic residue to a transition metal is important in synthesis. In compound II, the aromatic ring can be substituted at least with a group that can couple to a reactive dendrimer group, and with other groups that can modify the overall binding constant.

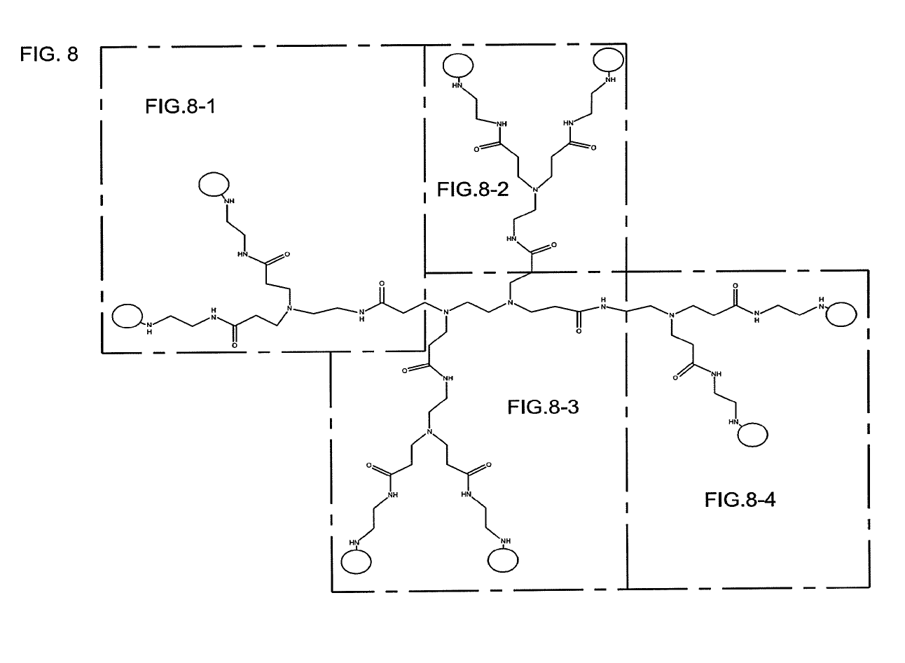

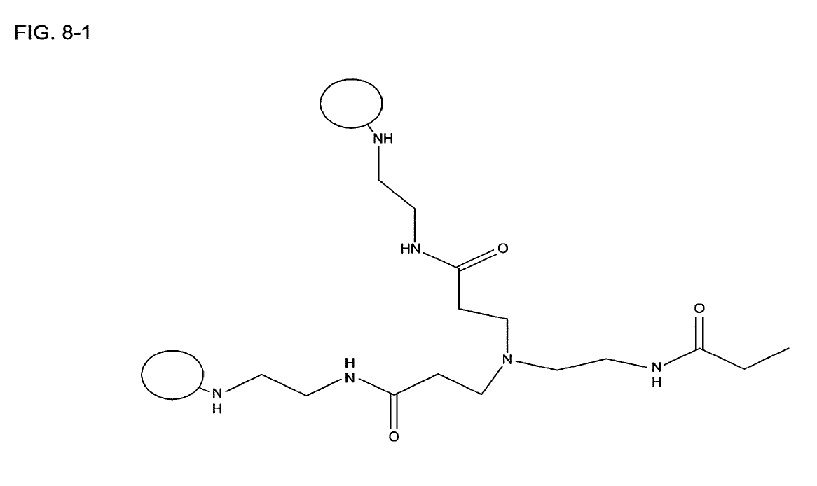

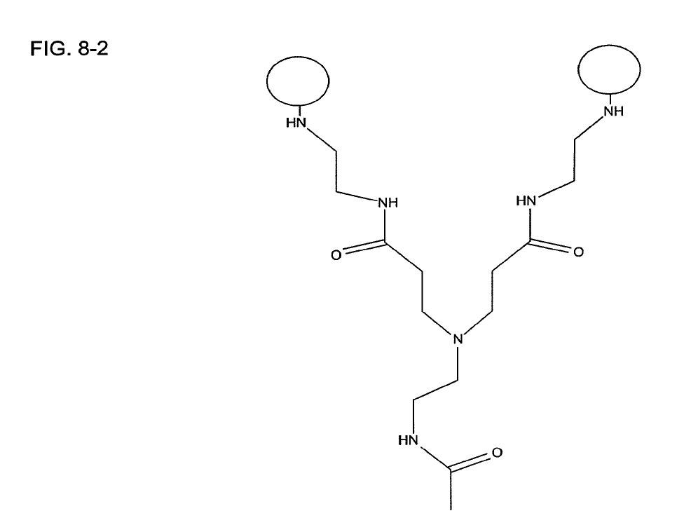

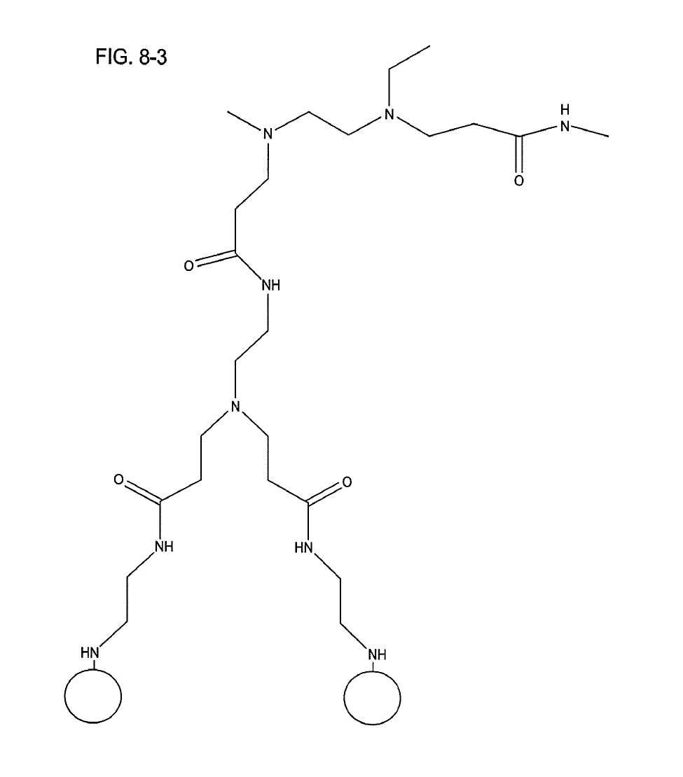

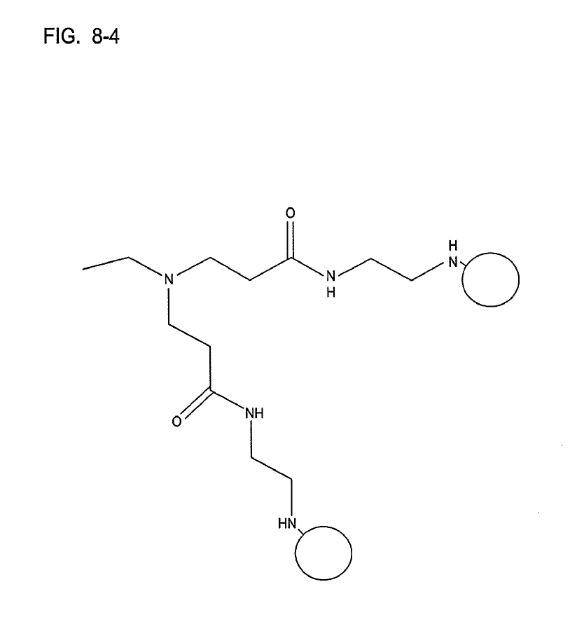

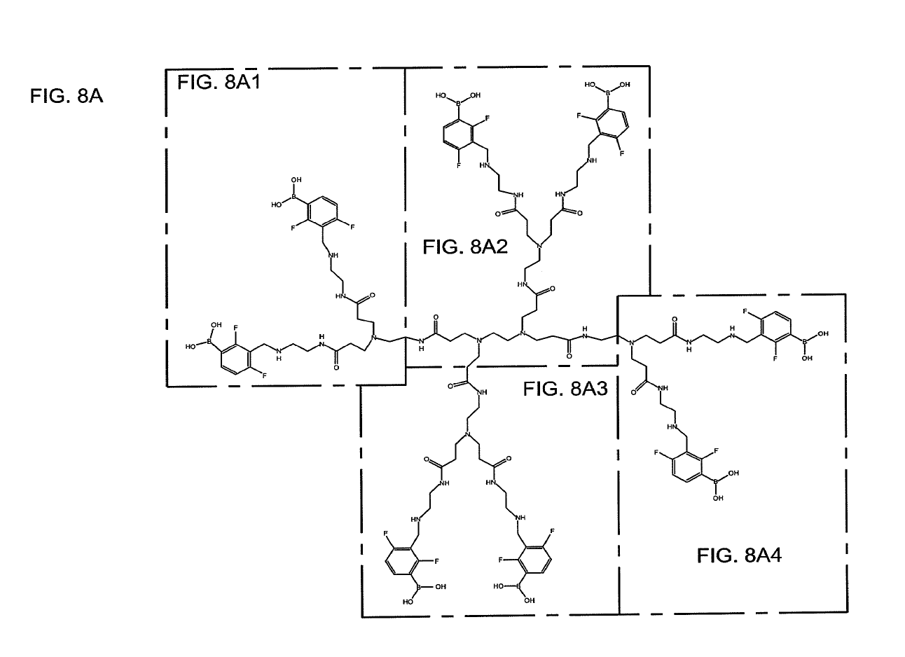

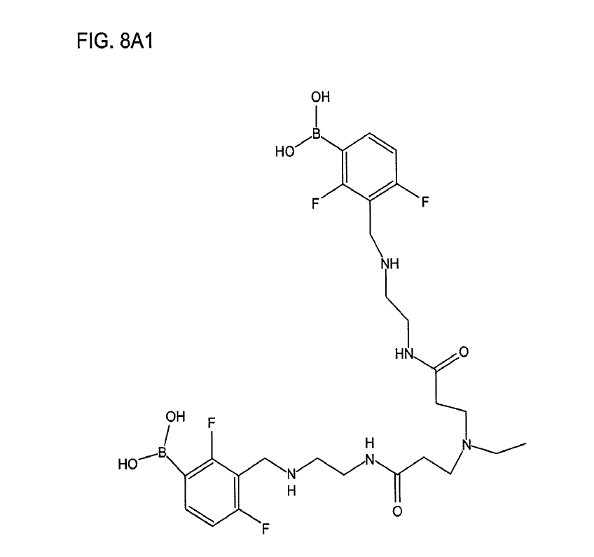

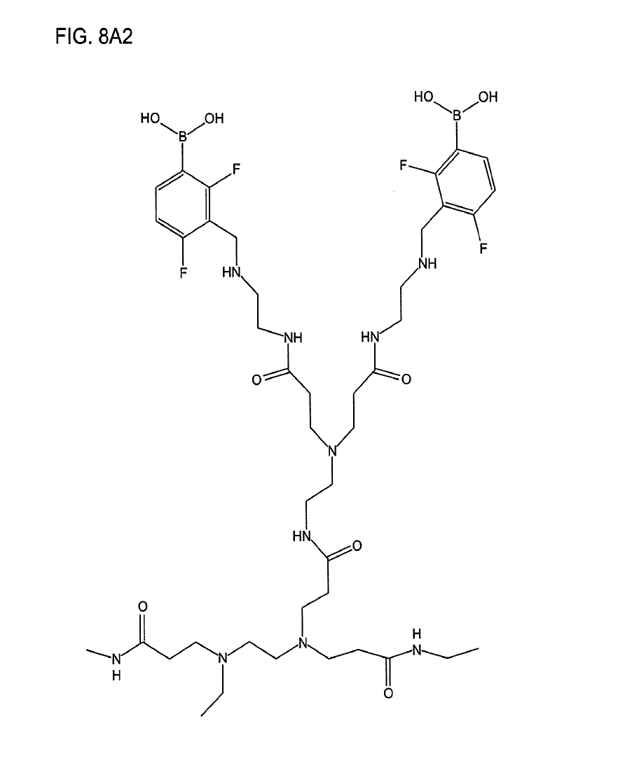

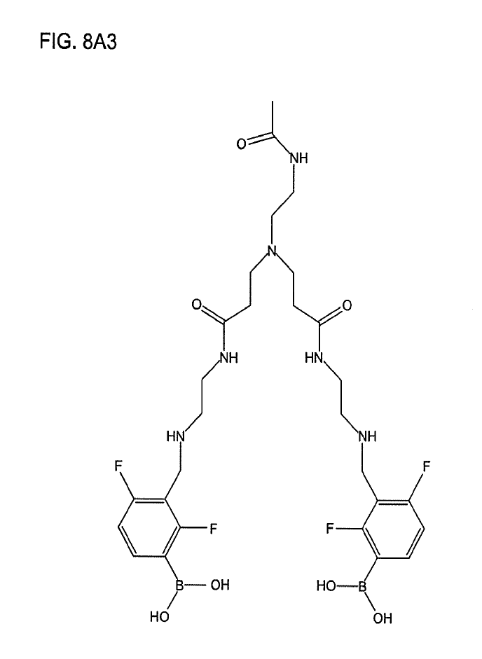

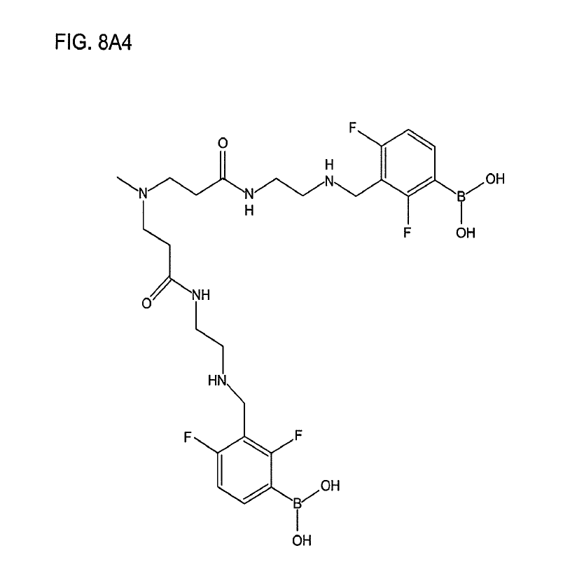

FIGS. 8 and 8A show two representations of one embodiment of the 6-1 dendrimer boronic acid component of the system.

In somewhat greater detail, boronic acids useful in the sensor are aromatic compounds such as:

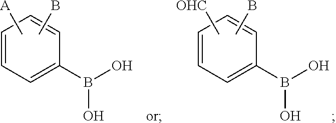

wherein B is a dendrimer reactive group and A comprises groups containing an oxygen, sulfur, amino, imino, or alkoxy; including such groups as hydrogen, halogen (such as fluoro- and chloro-), —CHO, —OH, —SH, —NH₂, —NHR₁, —N(R₁)₂, —CO₂H, —CO₂ R₁, —CO—NH₂, —CO—NH—R₁, —CO—N(R₁)₂, —CONH—NH₂, etc., In the above formula wherein each R or R₁ is independently alkyl of from 1 to 5 carbon atoms. The group —CHO is a formyl group.

A non-exhaustive listing of useful formyl aryl boronic acids is shown in Table 1.

TABLE 1
Aryl Boronic Acids

| | | |
|---|---|---|
| 1 | 2-Formylphenylboronic acid | (2-FPBA) |
| 2 | 3-Formylphenylboronic acid | (3-FPBA) |
| 3 | 4-Formylphenylboronic acid | (4-FPBA) |
| 4 | 2-Fluoro-3-formylphenyl boronic acid | (2-F-3-FPBA) |
| 5 | 2-Fluoro-4-formylphenylboronic acid | (2-F-4-FPBA) |
| 6 | 2-Fluoro-5-formylphenylboronic acid | (2-F-5-FPBA) |
| 7 | 3-Fluoro-5-formylphenylboronic acid | (3-F-5-FPBA) |
| 8 | 4-Fluoro-3-formylphenylboronic acid | (4-F-3-FPBA) |
| 9 | 5-Fluoro-2-formylphenylboronic acid | (5-F-2-FPBA) |
| 10 | 2,4-Difluoro-3-formylphenylboronic acid | (2,4-DF-3-FPBA) |
| 11 | 2,6-Difluoro-3-formylphenylboronic acid | (2,6-DF-3-FPBA) |
| 12 | 3,5-Difluoro-4-formylphenylboronic acid | (3,5-DF-4-FPBA) |
| 13 | 4-Borono-2-(tri-fluoromethyl)benzoic acid | (4-B-2-TFMBA)) |
| 14 | 3-Carboxy-5-nitrophenylboronic acid | (3-C-5-NPBA) |

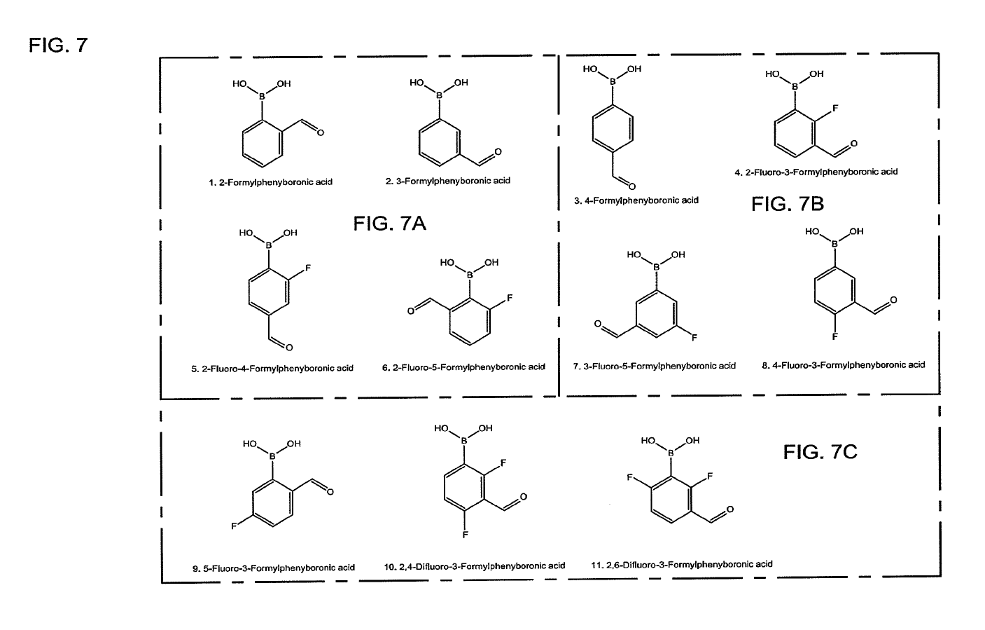

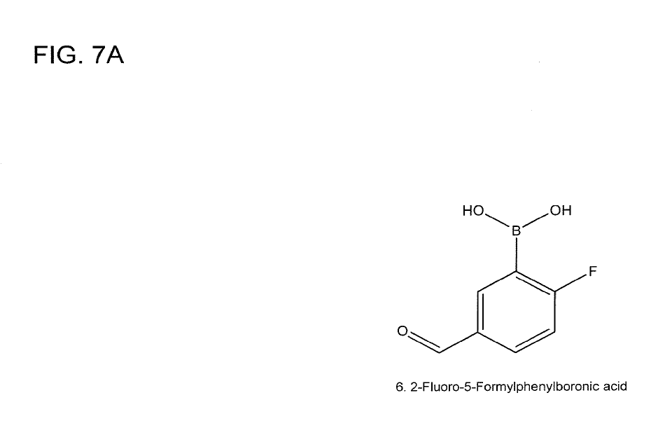

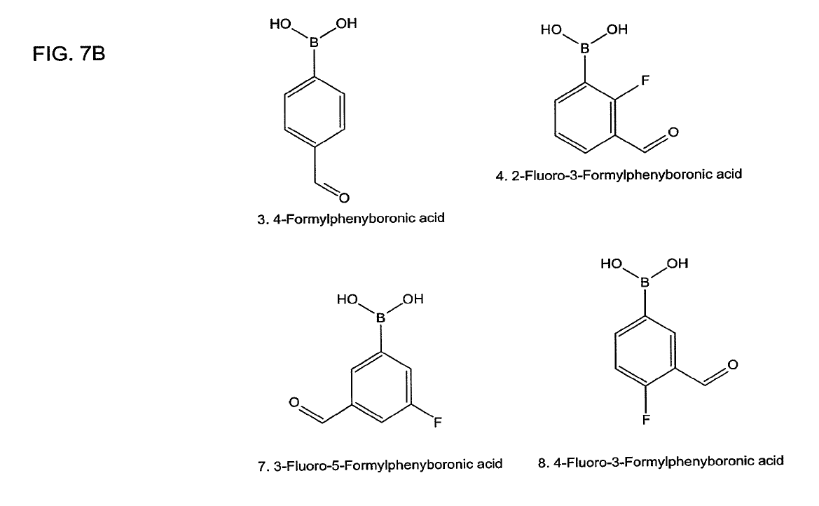

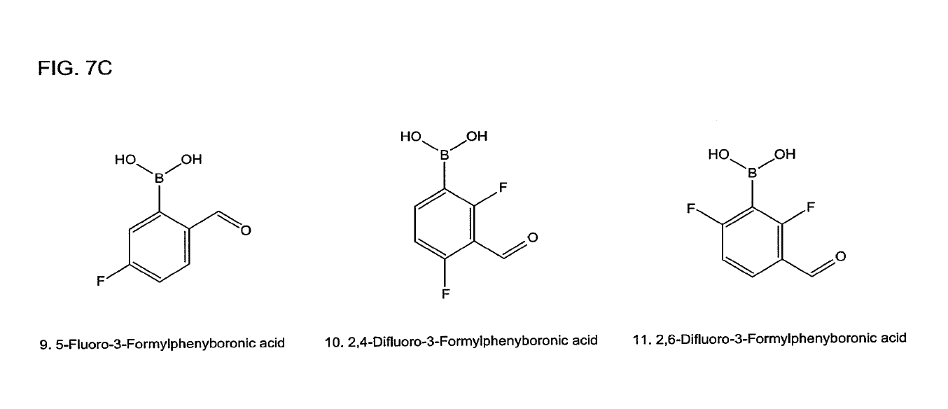

FIG. 7 has an array of useful boronic acid structures including ones listed in table 1. A non-exhaustive listing of useful dendrimer-aryl boronic acids components are shown in Table 2:

### Dendrimers

Three main considerations influenced the design of the DBA scaffold. These included: 1) selection of the appropriate scaffold to arrange the recognition motif in the correct orientation to support binding affinity and specificity, 2) selection of a scaffold with a mass sufficient to create a differential with glucose in order to generate a detectable signal, and 3) selection of a construct of appropriate size to prevent the signaling/competition component from diffusing out of the sensing system compartment.

Owing to their physical and chemical properties, dendrimers are advantageous for the construction of synthetic receptor materials and stable sensing applications. Dendrimers have a spherical and highly branched 3-D architecture that gives them a well-defined composition and topology. These characteristics, combined with their high-density surface functional group capacity for boronic acid immobilization, give dendrimers desirable physical, chemical and polyvalency characteristics. Their highly functionalized terminal surfaces also allow for control over the display of surface recognition elements. In addition, dendrimers are frequently exploited in physiological systems because they are water soluble, biocompatible and non-immunogenic. They are commercially available in a number of different generations and have size and mass characteristics that are compatible with our sensing system and implantable device design.

These characteristics make dendrimers ideally suited as scaffolds for the DBA competition/signaling component. They simultaneously provide a water soluble, stable and polyvalent scaffold that facilitates and stabilizes the conjugation of the otherwise insoluble and unstable boronic acid recognition moieties at the dendrimer surface.

Boronic acid analogues were selected for inclusion in the DBA library based on pre-screening of their interactions with our target analyte (glucose) versus. their interactions with our saccharide mimic diol compounds. Utilizing ARS, a diol selective fluorescent dye, we characterized the binding interactions of the initial kit of boronic acids with each diol species. Indicator displacement assays, such as the ARS assay, rely on the relative affinity of two competing guests for the receptor host. Specifically, the saccharide or diol-containing species, as the analyte of interest, competes with and preferentially displaces the diol-containing ARS from the boronic acid host. The displacement of the ARS reporter molecule from the boronic acid structure causes a measurable change in fluorescence. The magnitude of the fluorescence change that results from increasing concentrations of analyte provides a straightforward method to determine which boronic acid structures bind competitively with glucose and/or the saccharide mimics under the conditions (e.g. pH, ionic strength) of the assay.

ARS competitive assays were performed in a physiological buffer at pH 7 to confirm that the preselected kit of boronic acid ligands, which were selected to include a range of structural and chemical properties, bound glucose with adequate affinity in an aqueous environment. If the observed ARS fluorescence dropped substantially as the concentration of glucose titrated into the assay solution increased, we could conclude that glucose was competitive with the ARS diol relative to the boronic acid. In that case, the boronic acid was deemed to have passed our screening guidelines. On the other hand, if there was no observed change in fluorescence as increasing amount of glucose was titrated into the assay solution, we could conclude that glucose could not compete for the boronic acid with adequate affinity and, as a result, that particular boronic acid would no longer be considered as a viable candidate.

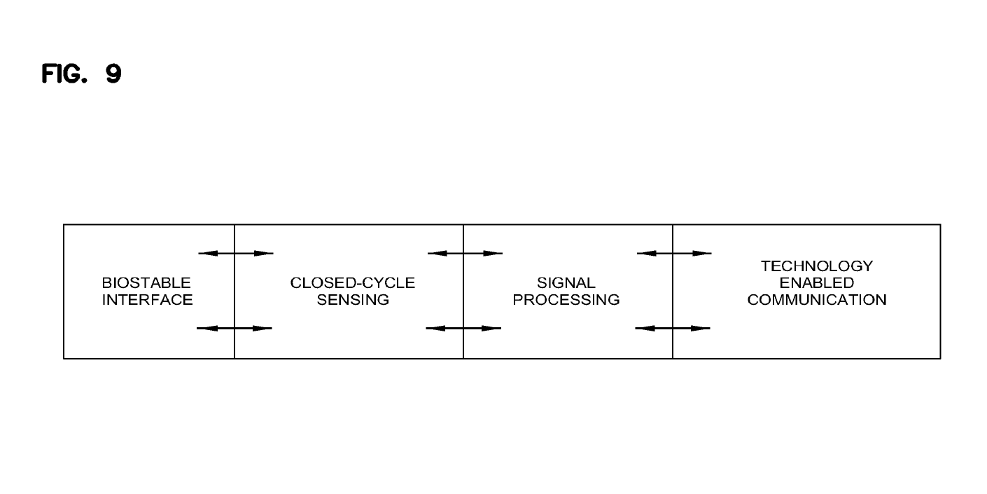

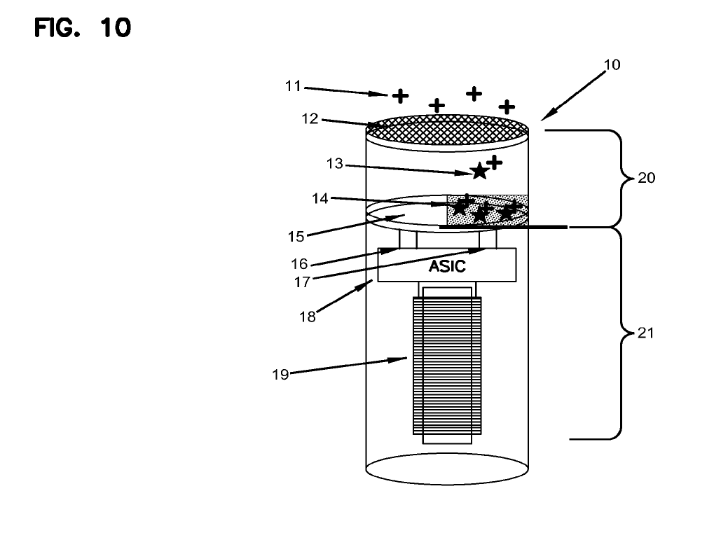

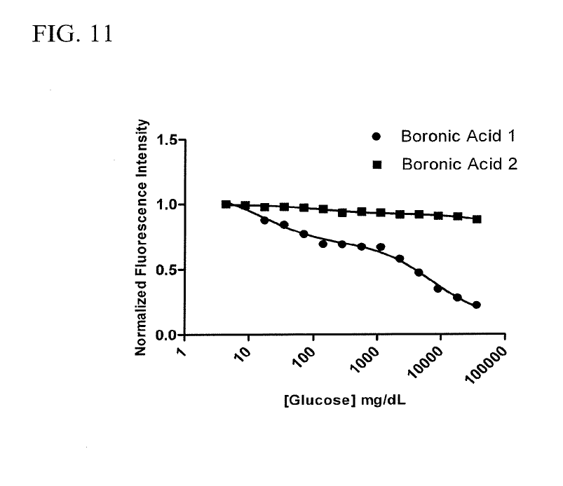

Response curves from a representative ARS assay experiment are shown in FIG. 11. The observed drop in fluorescence intensity as the concentration of glucose titrated into the solution increased demonstrated that glucose could bind to phenyl boronic acid 1 (FIG. 7C-10) and compete with ARS. In other words, the affinity of phenyl boronic acid 1 for glucose was greater than the affinity of phenyl boronic acid 1 for ARS, causing phenyl boronic acid 1 to preferentially bind with glucose. In contrast, phenyl boronic acid (FIG. 7C-11) showed little affinity toward glucose and was not included in construction of the DBA library. As predicted from structure-pKₐ relationships, phenyl boronic acid 1 would have greater binding affinity for glucose than for phenyl boronic acid 2. According to Hammet equation predictions, the quantifiable difference between phenyl boronic acid 1 and phenyl boronic acid 2 is the fluoro substituent located in the para-position on the phenylboronic acid structure. The electron withdrawing effect of the fluoro substituent in the para-position, on phenyl boronic acid 1, combined with a less sterically hindered boronic acid, will cause a drop in pKₐ and an increase in binding affinity to glucose.

Following boronic acid-glucose binding affinity pre-screening, we synthesized a library of DBAs using the selected, candidate boronic acids. Each DBA in the library was subsequently screened against each candidate saccharide mimic.

TABLE 2
Dendrimer-Boronic Acids (DBAs)

| | | |
|---|---|---|
| 1 | G1 + 2-Formylphenylboronic acid | G1 + 2-FPBA |
| 2 | G1 + 3-Formylphenylboronic acid | G1 + 3-FPBA |
| 3 | G1 + 4-Formylphenylboronic acid | G1 + 4-FPBA |
| 4 | G1 + 2-Fluoro-3-formylphenylboronic acid | G1 + 2-F-3-FPBA |
| 5 | G1 + 2-Fluoro-4-formylphenylboronic acid | G1 + 2-F-4-FPBA |
| 6 | G1 + 2-Fluoro-5-formylphenylboronic acid | G1 + 2-F-5-FPBA |
| 7 | G1 + 3-Fluoro-5-formylphenylboronic acid | G1 + 3-F-5-FPBA |
| 8 | G1 + 4-Fluoro-3-formylphenylboronic acid | G1 + 4-F-3-FPBA |
| 9 | G1 + 5-Fluoro-2-formylphenylboronic acid | G1 + 5-F-2-FPBA |
| 10 | G1 + 2,4-Difluoro-3-formylphenylboronic acid | G1 + 2,4-DF-3-FPBA |
| 11 | G1 + 2,6-Difluoro-3-formylphenylboronic acid | G1 + 2,6-DF-3-FPBA |
| 12 | G1 + 3,5-Difluoro-4-formylphenylboronic acid | G1 + 3,5-DF-4-FPBA |
| 13 | G1 + 4-Borono-2-(trifluoromethyl)benzoic acid | G1 + 4-B-2-TFMBA |
| 14 | G1 + 3-Carboxy-5-nitrophenylboronic acid | G1 + 3-C-5-NPBA |
| 15 | G2 + 2-Formylphenylboronic acid | G2 + 2-FPBA |
| 16 | G2 + 3-Formylphenylboronic acid | G2 + 3-FPBA |
| 17 | G2 + 4-Formylphenylboronic acid | G2 + 4-FPBA |
| 18 | G2 + 2-Fluoro-3-formylphenylboronic acid | G2 + 2-F-3-FPBA |
| 19 | G2 + 2-Fluoro-4-formylphenylboronic acid | G2 + 2-F-4-FPBA |
| 20 | G2 + 2-Fluoro-5-formylphenylboronic acid | G2 + 2-F-5-FPBA |
| 21 | G2 + 3-Fluoro-5-formylphenylboronic acid | G2 + 3-F-5-FPBA |
| 22 | G2 + 4-Fluoro-3-formylphenylboronic acid | G2 + 4-F-3-FPBA |
| 23 | G2 + 5-Fluoro-2-formylphenylboronic acid | G2 + 5-F-2-FPBA |
| 24 | G2 + 2,4-Difluoro-3-formylphenylboronic acid | G2 + 2,4-DF-3-FPBA |
| 25 | G2 + 2,6-Difluoro-3-formylphenylboronic acid | G2 + 2,6-DF-3-FPBA |
| 26 | G2 + 3,5-Difluoro-4-formylphenylboronic acid | G2 + 3,5-DF-4-FPBA |
| 27 | G2 + 4-Borono-2-(trifluoromethyl)benzoic acid | G2 + 4-B-2-TFMBA |
| 28 | G2 + 3-Carboxy-5-nitrophenylboronic acid | G2 + 3-C-5-NPBA |
| 29 | G3 + 2-Formylphenylboronic acid | G3 + 2-FPBA |
| 30 | G3 + 3-Formylphenylboronic acid | G3 + 3-FPBA |
| 31 | G3 + 4-Formylphenylboronic acid | G3 + 4-FPBA |
| 32 | G3 + 2-Fluoro-3-formylphenylboronic acid | G3 + 2-F-3-FPBA |
| 33 | G3 + 2-Fluoro-4-formylphenylboronic acid | G3 + 2-F-4-FPBA |
| 34 | G3 + 2-Fluoro-5-formylphenylboronic acid | G3 + 2-F-5-FPBA |
| 35 | G3 + 3-Fluoro-5-formylphenylboronic acid | G3 + 3-F-5-FPBA |
| 36 | G3 + 4-Fluoro-3-formylphenylboronic acid | G3 + 4-F-3-FPBA |
| 37 | G3 + 5-Fluoro-2-formylphenylboronic acid | G3 + 5-F-2-FPBA |
| 38 | G3 + 2,4-Difluoro-3-formylphenylboronic acid | G3 + 2,4-DF-3-FPBA |
| 39 | G3 + 2,6-Difluoro-3-formylphenylboronic acid | G3 + 2,6-DF-3-FPBA |
| 40 | G3 + 3,5-Difluoro-4-formylphenylboronic acid | G3 + 3,5-DF-4-FPBA |
| 41 | G3 + 4-Borono-2-(trifluoromethyl)benzoic acid | G3 + 4-B-2-TFMBA |
| 42 | G3 + 3-Carboxy-5-nitrophenylboronic acid | G3 + 3-C-5-NPBA |
| 43 | G4 + 2-Formylphenylboronic acid | G4 + 2-FPBA |
| 44 | G4 + 3-Formylphenylboronic acid | G4 + 3-FPBA |
| 45 | G4 + 4-Formylphenylboronic acid | G4 + 4-FPBA |
| 46 | G4 + 2-Fluoro-3-formylphenylboronic acid | G4 + 2-F-3-FPBA |
| 47 | G4 + 2-Fluoro-4-formylphenylboronic acid | G4 + 2-F-4-FPBA |
| 48 | G4 + 2-Fluoro-5-formylphenylboronic acid | G4 + 2-F-5-FPBA |
| 49 | G4 + 3-Fluoro-5-formylphenylboronic acid | G4 + 3-F-5-FPBA |
| 50 | G4 + 4-Fluoro-3-formylphenylboronic acid | G4 + 4-F-3-FPBA |
| 51 | G4 + 5-Fluoro-2-formylphenylboronic acid | G4 + 5-F-2-FPBA |
| 52 | G4 + 2,4-Difluoro-3-formylphenylboronic acid | G4 + 2,4-DF-3-FPBA |
| 53 | G4 + 2,6-Difluoro-3-formylphenylboronic acid | G4 + 2,6-DF-3-FPBA |
| 54 | G4 + 3,5-Difluoro-4-formylphenylboronic acid | G4 + 3,5-DF-4-FPBA |
| 55 | G4 + 4-Borono-2-(trifluoromethyl)benzoic acid | G4 + 4-B-2-TFMBA |
| 56 | G4 + 3-Carboxy-5-nitrophenylboronic acid | G4 + 3-C-5-NPBA |

Polyamidoamine (PAMAM) Dendrimers

PAMAM dendrimers are “dense star” polymers that provide a unique macromolecular architecture useful for polyvalent binding. These starburst dendrimers are formed using a stepwise polymerization process that is used to control the shape, density and surface functional groups. Dendrimers are comprised of a central core (in our case an ethylenediamine-core) that is capped with repeat units, layer-by-layer, of branched “arms” or internal structures that branch radially outward from the core. As a layer of repeat unit is added to the central core, the generation number increases. With each generation, the MW more than doubles and the number of unique surface or terminal primary amine groups exactly doubles (see table below). Some other desirable properties of these macromolecular structures: control over type and display or the surface recognition elements, aqueous solubility, narrow MW distribution, high degree of molecular uniformity, monodisperse, globular.

TABLE 3
PAMAM Dendrimer

| | Molecular | | Surface |
|---|---|---|---|
| | Weight | Diameter | Functional |
| Generation | (g/mol) | (Angstroms) | Groups |
| 1 | 1430 | 22 | 8 |
| 2 | 3256 | 29 | 16 |
| 3 | 6909 | 36 | 32 |
| 4 | 14215 | 45 | 64 |

The PAMAM dendrimers used in our experiments were purchased from Dendritech, Inc. (Midland, Mich.). We used only G1 through G4, as shown in the table above. There are higher generations available with different terminal functional groups. Also, this information found in the table is from Dendritech, Inc.

For each generation, we are able to attach the number of boronic acids as a functional group that are permitted via the primary amine surface functional groups. For example, with a generation 1 (G1) dendrimer, we can attach 8 boronic acids as functional groups to bond to the immobilized iDIOL. With a generation 2 (G2) dendrimer, we can attach 16 boronic acids. And so on. Of course you can manipulate the number of boronic acids that can be attached (ex. half load or quarter load, etc.) by controlling the equivalents or blocking.

### Polyol-iDIOL

Polyols, in the form of immobilized saccharide mimics (iDIOLs), have been used by our group as a glucose-competitive, DBA-binding environment in a competitive binding assay that serves as the prototype for the ultimate mass-sensitive, in vivo glucose sensor. Thus, the second critical step required for the demonstration of the self-contained glucose sensing system was the selection of the glucose-competitive DBA binding environment (iDIOL). The selection of materials for this component were governed by the need to: 1) construct a glucose-competitive binding environment that would form a reversible complex with the DBA signaling component in aqueous media and 2) select commercially available saccharide mimics with a diversity of diol sub-structures and a suitable functional moiety for covalent immobilization to a support.

This iDIOL versus DBA strategy, as discussed in detail earlier, uses the observation that the hydroxyl groups on saccharides, specifically 1,2- or 1,3-diols, competitively bind with boronic acids to form five- or six-membered ring structures. We initially selected diols, which would subsequently be immobilized to produce the required iDIOLs, based on a comparison of their binding affinity to DBAs versus the binding affinity of the respective DBA for glucose. Our diol selection strategy involved exploiting the differential in relative binding affinity that would be created when a DBA is concurrently exposed to an immobilized diol (iDIOL) and a range of glucose concentrations. The objective was to identify DBA:iDIOL pairs that would permit discriminatory binding of the DBA to glucose, due to increased relative affinity over DBA binding to the iDIOL.

Selection of diols for ultimate preparation of iDIOLs, via immobilization of the diol on the sensing system's transduction interface, was based on our evaluation of the interactions between our kit of boronic acid-derived DBAs and various candidate diol species. Any detection system can be used to detect selective binding. We again used the ARS assay, as described previously, to characterize the binding of the DBAs with the candidate diols as a proof of concept. The magnitude of the change in ARS fluorescence (any electrical, mechanical, or chemical change can be used) that resulted from increasing the amount of diol titrated into the assay solution provided a straightforward method to determine which iDIOLs, and ultimately which iDIOL structures, would competitively interact with the various DBA species.

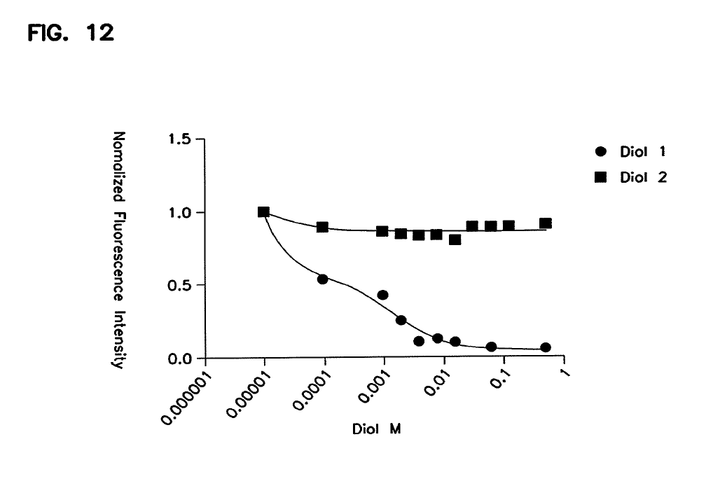

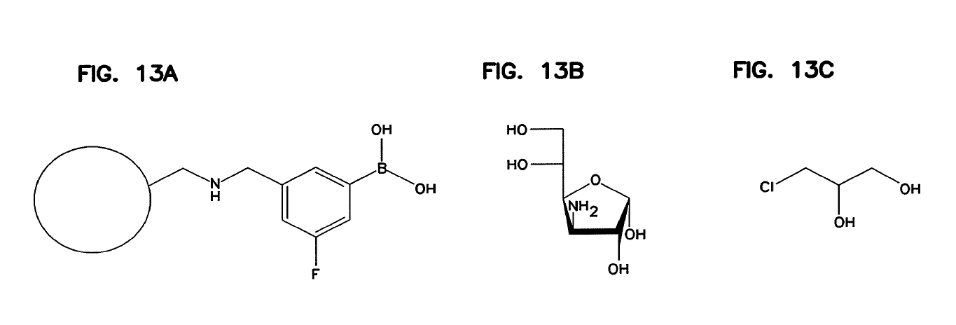

Response curves from an ARS assay experiment performed in a physiological buffer at neutral pH are shown in FIG. 12. An enhanced response of the DBA 2 (FIG. 13.A) for diol 1 (FIG. 13.B) versus diol 2 (FIG. 13.C) was observed. Phenylboronic acids are known to have different binding affininites for diols depending on the dihedral angle of the diol. Smaller dihedral angles often accompany higher binding constants. Additionally, rigid cyclic cis diols tend to form stronger cyclic esters than acyclic diols. Thus, the enhanced binding of diol 1 can be attributed to the improved compatibility of the boronic acid recognition motif on DBA 2 with the dihedral angle of the diol. In contrast, it can be inferred that diol 2 formed a weaker cyclic ester with the same boronic acid of DBA 2 as a result of increased angle strain of the larger dihedral angle structure of the acyclic diol.

The drop in fluorescence intensity as the concentration of diol titrated into the solution increased demonstrated that diol 1 could bind to the DBA with an affinity sufficient to release the DBA from the DBA:ARS complex. By contrast, the DBA showed little affinity towards diol 2, which was subsequently not considered for immobilization as an iDIOL. Based on the results of this screening process, we generated a library of diols that, when immobilized as iDIOLs, encompassed a range of DBA:diol and DBA:glucose binding affinities. The database of diol/iDIOL chemical and physical properties, as they related to binding affinity, became part of the toolbox that enabled us to screen for the optimal signaling component relative to the desired glucose-competitive DBA binding environment.

In order to achieve identification of lead DBA:iDIOL pairs for subsequent evaluation as candidate glucose sensing system components, we required a method that would allow us to determine the relative affinities of DBA:glucose versus DBA:diol. As a consequence, we began systematically evaluating the K_{eq} values of DBA:diol and DBA:glucose candidates using their ARS profiles, over a range of diol/glucose concentrations. Although it could be viewed as necessary to screen every potential candidate DBA:diol combination to determine their response to glucose, even a limited set of boronic acids (e.g. n=50) incorporated into a series of dendrimer generations as DBA constructs (e.g. n=5) and evaluated against iDIOL candidates (e.g. n=50) gives a formidable number (e.g. n=50×5×50=12,500) of possible combinations. In order to overcome the technical and resource challenges of such a laborious screening process, we built a binding affinity model and database based on a three-component DBA:glucose:diol interaction model. Establishing a foundation based on an affinity model database was critical to furthering our efforts toward designing a system whose function relies on the affinities of the sensing system components. These derived K_{eq} values were used to identify lead DBA and diol candidates. By comparing K_{eq} values, we were able to estimate how sensitively each DBA would respond to glucose and identify components that would best fit a sensing system designed to detect glucose over the physiological range. Not only did this approach significantly limit the number of DBA:iDIOL candidate combinations that would need to be screened, it quantified and allowed us to directly compare binding between each DBA:glucose and DBA:diol pair.

Experimental K_{eq} values of DBA:diol and DBA:glucose combinations were generated utilizing the three-component competitive assay developed by Springsteen and Wang. Using ARS as the fluorescent reporter, the association constant between each respective DBA:glucose and DBA:diol pair was determined. Within this system there are two competing equilibria, the first between the candidate DBA and the ARS reporter and the second between the candidate DBA and glucose or saccharide mimic diol. Fluorescence intensity changes, as they relate to the formation and perturbation of each equilibria, were used to calculate the K_{eq} of glucose and the diol relative to the DBA. These data were ranked according to the magnitude of the K_{eq} to facilitate selection of DBA(s) for use as competition signaling components and diols(s) for immobilization as iDIOL binding environments.

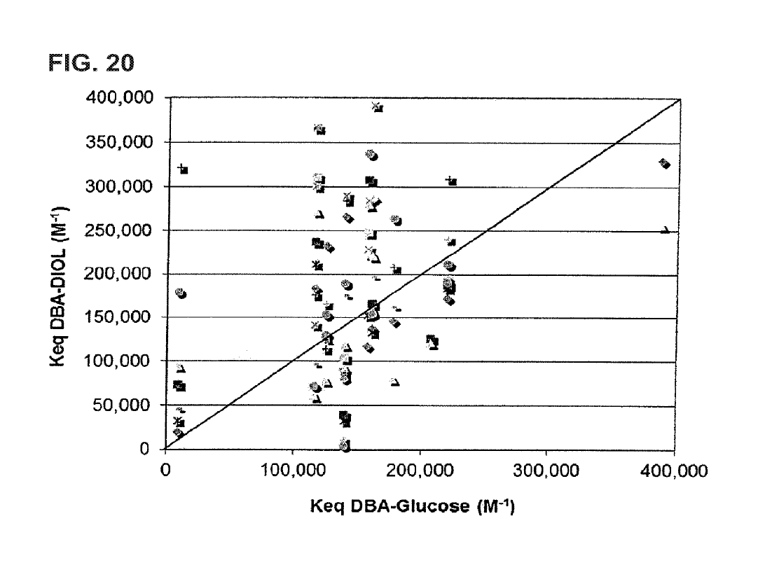

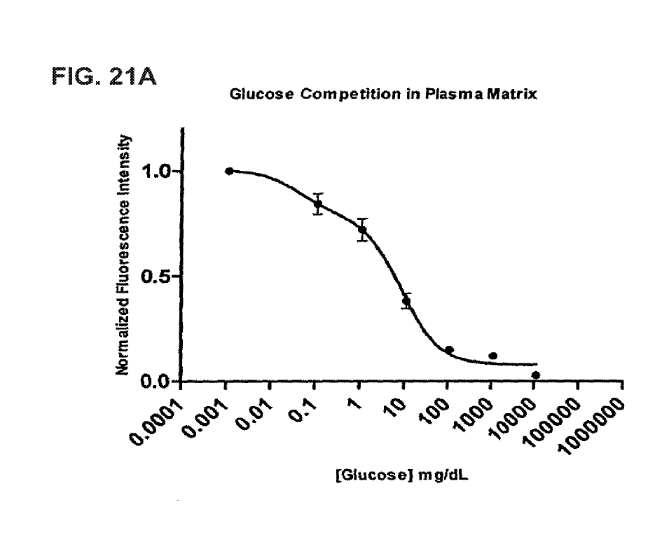

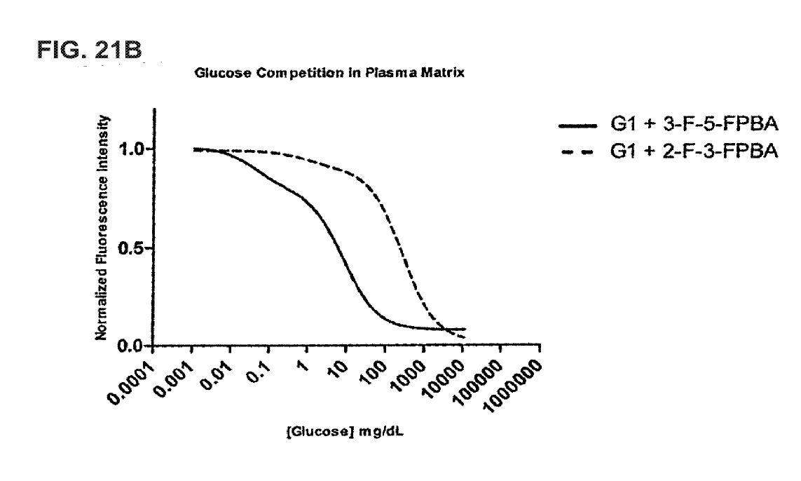

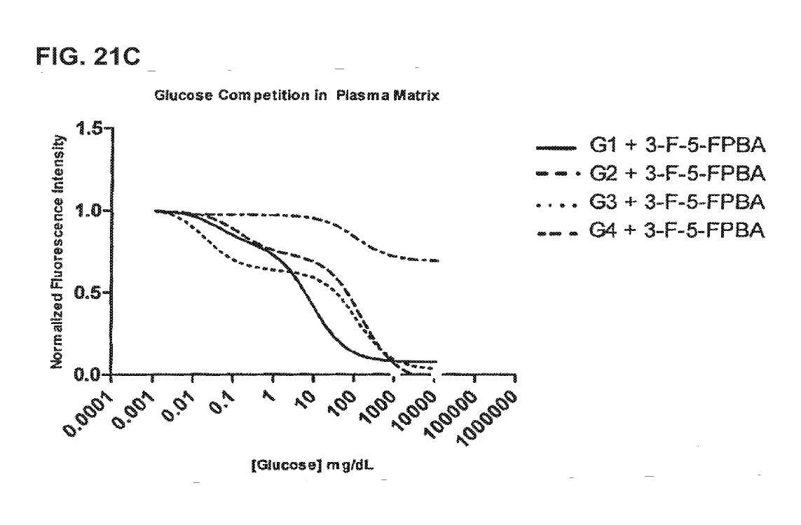

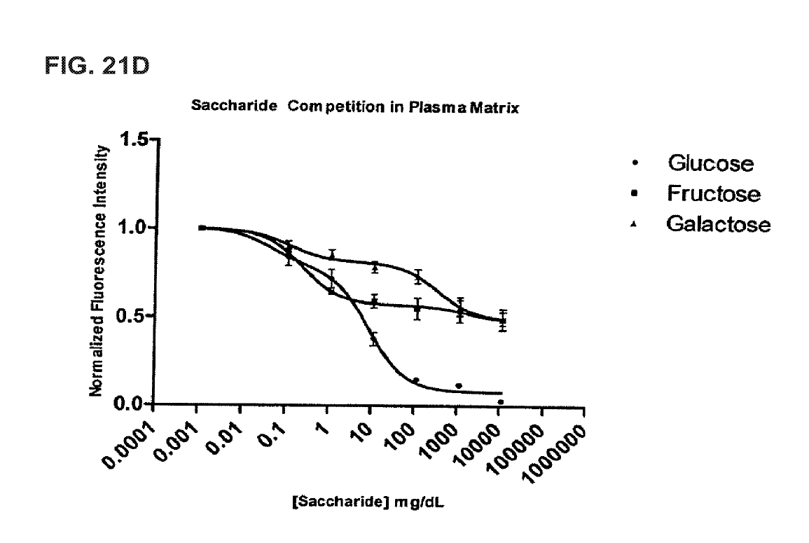

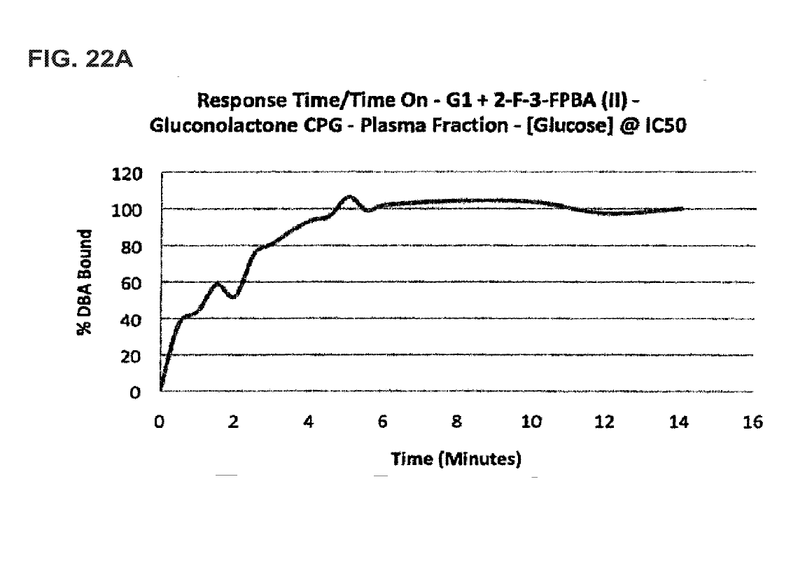

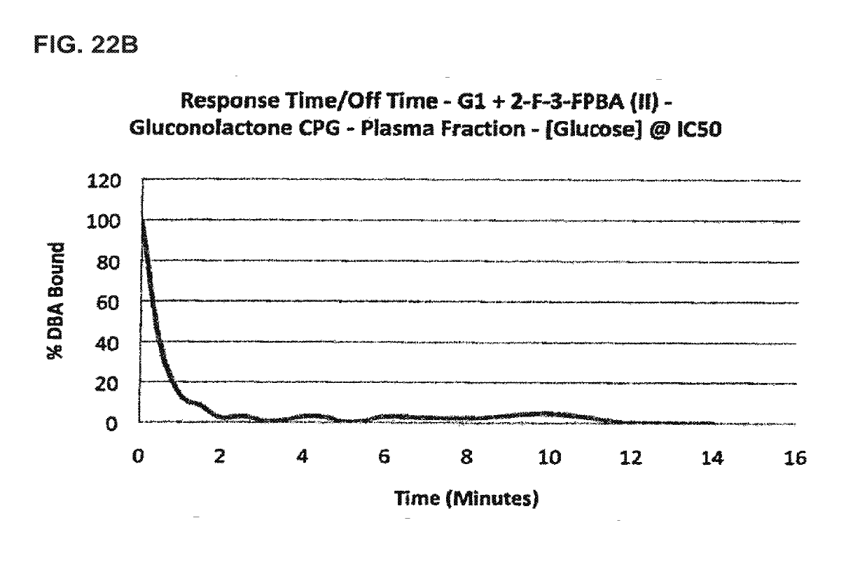

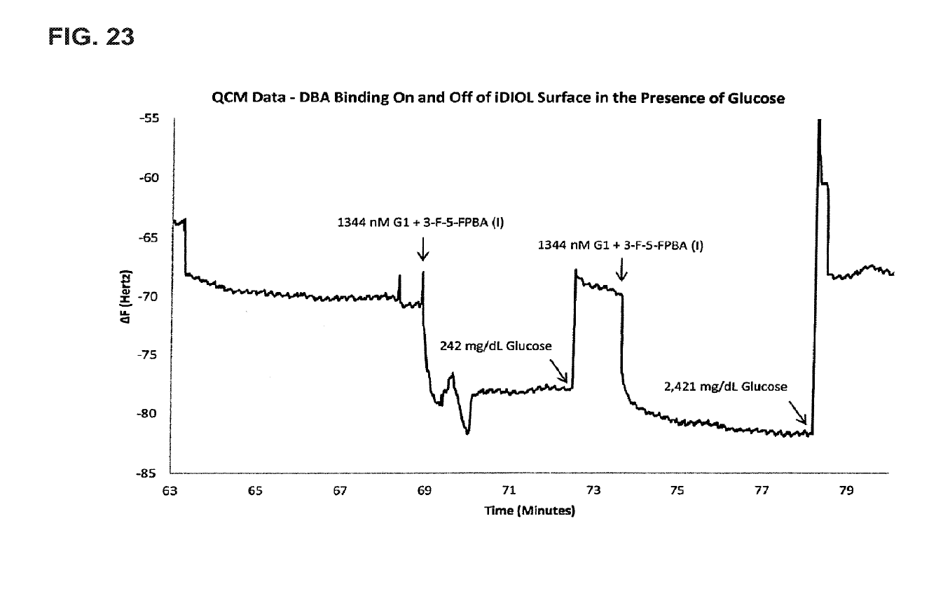

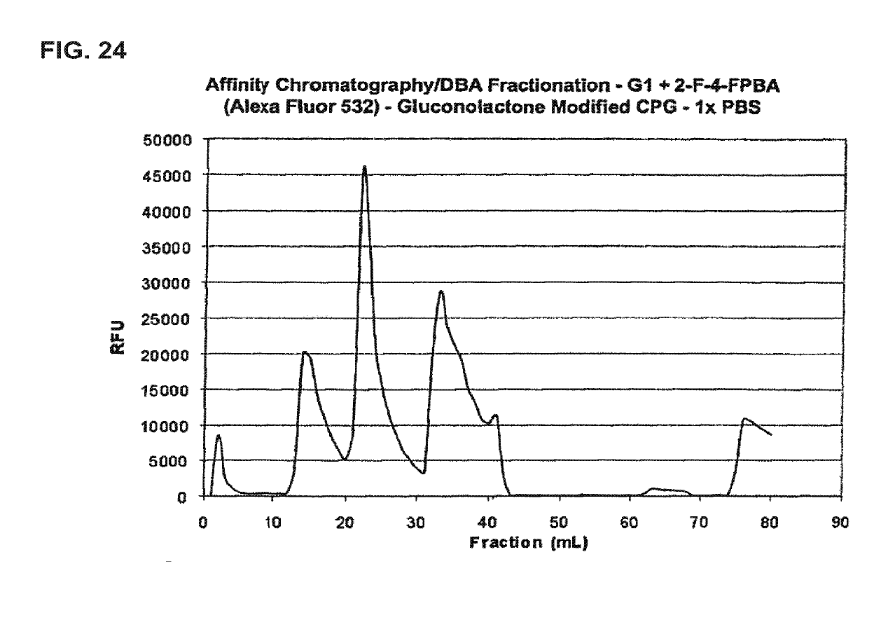

Keq values of each DBA:diol and DBA:glucose combination provides a wide range of relative affinities encompassed in our DBA and saccharide mimic libraries. Based on the location of a representative data point on the interaction graph, the relative affinity of glucose versus each diol for that DBA can be easily compared. For example, if a data interaction point is located along the 1:1 line, as depicted in FIG. 20, this indicates that the relative binding affinity of the candidate DBA for glucose is similar to the binding affinity of the same DBA for the diol of the DBA:diol pair. Additionally, if a data interaction point is located along the 2:1 line, the binding strength of the candidate DBA for glucose is approximately twice the binding strength of the same DBA for the diol. This may signify that a data interaction point on the 2:1 line represents a DBA:diol that is more sensitive to glucose than a DBA:diol pair on the 1:1 line. Depending on how the binding affinity values for a DBA:glucose and DBA:diol pair differed in magnitude, that particular DBA:diol pair was either eliminated or included as a lead pair in further glucose competition assay screening experiments.

Our libraries of DBA and diol compounds were systematically evaluated for K_{eq} under conditions (ionic species, pH, etc.) that resembled those of an in vivo environment. This data system was designed as a guide to rapidly compare relative binding affinities of a large number of DBA and diol species before committing to diol immobilization as an iDIOL environment and subsequent DBA:glucose:iDIOL surface competition screening. Significantly, data extrapolated from the K_{eq} interaction graph streamlined our efforts in estimating how each DBA:iDIOL combination would respond to glucose.

The system of the invention includes a polyol. The polyol is a generally hydrophilic compound. As polyol compounds, there may be mentioned hydrophilic polyols that include glycerin, poly(vinyl alcohol), poly(ethylene glycol), polypropylene glycol, etc.). Other polyols include oligo-, di- and monosaccarides such as sucrose, marmitol, lactose, L-arabinose, D-erythrose, D-ribose, D-xylose, D-marmose, D-galactose, lactulose, cellobiose, gentibiose, etc. Preferred polyols are natural or synthetic small molecule polyol or saccharide compounds. FIG. 6 shows an array of useful polyols including conventional small molecule polyols including industrial and saccharide compounds that can be immobilized to the surface in the system of the invention. The —OH group of the polyol must be available on the surface to reversible bind to either the DBA or the analyte.

### Useful-

### Polyols

### Immobilized

as iDIOLS
iDIOL 1-(1S,2R,3S,4S)-2,3-Dihydroxy-4-(hydroxymethyl)-1-aminocyclopentane hydrochloride (ACROS, 98% ee)

### 2-Chloroethyl-b-D-fructopyranoside (Carbosynth, Ltd.)

iDIOL 2-3-Chloro-1,2-propanediol

### (Sigma)

Valiolamine hydrate (Carbosynth, Ltd.)
iDIOL 3-Gluconolactone
(−)-2,3:4,6-Di-O-isopropylidene-2-keto-L-gulonic acid monohydrate*(Sigma)

### Detection System

Any system that can detect the relative binding competition as disclosed can be used. These systems include mechanical, electrical and chemical, the competitive binding (and K_{eg}) are transmitted to a receiver by sending directly or converting the mechanical, chemical, or electrical signal into a signal that can be sent to a receiver. The signal is typically a frequency that varies in proportion to binding. We have found that systems using a change in fluorescence or mechanical frequency can be used.

### Fluorescent

### Fluorescent System of the Invention

We have found that a fluorescent molecule can be covalently coupled to the dendrimer structure component of the invention. A fluorescent molecule can be selected such that the fluorescence of the molecule is either enhanced or quenched as a fluorescent/dendrimer component is displaced from the immobilized polyol. Since the degree of fluorescent enhancement or quenching is proportional to the analyte quantity, the change in fluorescence, once equilibrium is reached, can indicate the concentration of the analyte. A fluorophore, in analogy to a chromophore, is a component of a fluorescent molecule which causes that molecule to be fluorescent. A fluorophore is a functional group in a molecule which will absorb energy of a specific wavelength and re-emit energy at a different (but equally specific) wavelength. The amount and wavelength of the emitted energy depend on both the fluorophore and the chemical environment of the fluorophore. This technology has particular importance in the field of biochemistry and protein studies, e.g., in immunofluorescence and immunohistochemistry. Fluorescent compounds are known and the useful compounds include those that can be coupled to the dendrimer component preferably with a covalent bond. Useful fluorescent compounds include the following non-exhaustive listing: Alexa Fluor® 350; Alexa Fluor® 405; Alexa Fluor® 430; Alexa Fluor® 488; Alexa Fluor® 532; Alexa Fluor® 546; Alexa Fluor® 555; Alexa Fluor® 568; Alexa Fluor® 594; Alexa Fluor® 610; Alexa Fluor® 633; Alexa Fluor® 635; Alexa Fluor® 647; Alexa Fluor® 660; Alexa Fluor® 680; Alexa Fluor® 700; Alexa Fluor® 750; Alexa Fluor® 790; Allophycocyanin (APC); 6-Carboxyfluorescein (FAM); Cy® 2; Cy® 3; Cy® 5; Cy® 7; Fluorescein Isothiocyanate (FITC); Hexachlorofluoroscein (HEX); Rhodamine (TRITC); R-phycoerythrin (PE); Tetrachlorofluorescein (TET); Tetramethylrhodamine (TAMARA); and others similar in excitation and fluorescent properties.

### Piezoelectric

### Piezoelectric Cantilever Structure of the Invention

Microelectronic piezoelectric cantilever structures are known. These structures are made using electronic and semiconductor fabrication technology. The cantilever structure useful in the invention is piezoelectric such that an alternating current is placed across the structure causing the structure to have a stable frequency output. Such a cantilever can have immobilized on the cantilever surface, a binding group comprising an immobilized polyol compound of the invention. The polyol structure can be reversibly bonded to by the dendrimer-boronic acid component of the invention in competition with the analyte. The mass of the dendrimer-boronic acid reversibly bonded to the cantilever component affects the frequency of the output of the microelectronic piezoelectric cantilever structure. When used as a part of the system of the invention, the analyte competes with the dendrimer-boronic acid component (see FIG. 3). As the dendrimer-boronic acid component is displaced by the analyte, the dendrimer-boronic acid is in equilibrium between that bound to the iDIOL and the free, in solution DBA which then has some proportion of the boronic acid groups bound to the glucose analyte. As the glucose concentration increases, more of the free boronic acid groups are bound to analyte so that they are no longer available to bind to the iDIOL, as the concentration increases and the process continues this results in the complete displacement of the DBA from the iDIOL surface. The mass thereof leaves the surface of the cantilever structure, the mass on the cantilever changes due to the larger molecular size of the dendrimer structure relative to the (glucose) analyte. This process works for any analyte that is either lighter or heavier than the DBA, it then becomes a sensitivity issue. Since the frequency of the piezoelectric portion of the cantilever structure is proportional to the mass of the dendrimer-boronic acid on the cantilever structure, the frequency then changes as the mass changes. As a result, once equilibrium is reached, the final frequency difference indicates the concentration of the analyte.

### Container/Membrane

### Subcutaneous Container Structure

The system of the invention can be incorporated into a subcutaneous real-time monitoring sensor. In the construction of such a sensor, the system can be included within an analyte selective membrane. Such a membrane can entirely envelope the system or the system can be included in a permeable or unpermeable container having an opening which is sealed by the membrane. In operation, the membrane excludes materials that can interfere with the detection or analysis of the analyte. One embodiment is a molecular weight cut off membrane that can permit the entry of an appropriately sized analyte such as glucose into the sensor. Within the sensor, the analyte then appropriately competes with the dendrimer-boronic acid component with little or no non-target or non-specific interference, and produces a useful and detectable signal. Such a membrane can also maintain the large dendrimer-boronic acid structure within the sensor. Alternatively, the dendrimer-boronic acid can be chemically tethered to the sensor internal surfaces in such a way that the dendrimer-boronic acid is maintained available for the competitive reaction. When tethered by a flexible chain, the dendrimer-boronic acid can be in an off mode and its mass is not seen by the cantilever. Also, the fluorescent moiety can be maintained out of the excitation light zone. In a fluorescent mode, the detector can contain a photosensitive device that can quantify fluorescence that typically arises through a visible wavelength. Alternatively, the sensor can contain the piezoelectric cantilever structure that can provide a signal in proportion to analyte concentration.

Reversibly attached to the immobilized polyol are dendrimer-boronic acid components. In operation, the glucose from the patient penetrates the membrane which excludes high molecular weight materials. The exclusion of high molecular weight materials reduces the tendency of the piezoelectric sensor to provide a false read out. Within the cell, the glucose derived from the patient enters the test cell and competes with the dendrimer-boronic acid materials at or near the piezoelectric cantilever surface. Since glucose has a molecular weight substantially less than the dendrimer-boronic acid structures on the piezoelectric cantilever structure, the mass on the piezoelectric cantilever structure changes in proportion to glucose concentration as the mass of the dendrimer-boronic acid changes on the piezoelectric cantilever structure. As the mass drops, the resonant frequency of the piezoelectric cantilever changes. As the frequency changes, the potential output from the piezoelectric cantilever also changes. That change in electrical potential can be read as inversely proportional to the glucose concentration. Since the frequency of vibration of the piezoelectric sensor increases with reduced mass, the electrical output of the piezoelectric sensor provides a direct indication of the glucose concentration from the patient's arterial or peripheral blood, ascites, interstitial fluids, or other fluids in a subcutaneous space or zone of the body.

The subcutaneous analyte detector system has as one component a molecular weight cut off membrane. The purpose of the membrane is to permit the small molecule analyte to penetrate the membrane. Separating the analyte from other materials in the tested fluid can improve the test. In the instance that the patient has interfering compounds in the tested fluid, the membrane can reduce interference from higher molecular weight materials. Once inside the device, the analyte can then interact with the treated cantilever structure generating a signal in proportion to the concentration of the analyte. The molecular weight cut off membrane is selected such that the analyte is available for analysis and that the dendrimer-boronic acid is maintained in the sensor. The molecular weight cut off membrane (MWCO) typically is formed of a material having a pore size that is designed to act as a molecular weight cut off mechanism. The molecular weight of MWCO is typically measured in daltons (Da). The molecular weight cut off can be typically greater than 500 Da, often greater than 1,000 Da, and typically greater than 10,000 Da or higher.

A useful membrane can be made from a variety of materials as long as the material can have a pore size or the correct molecular weight cut off. Typical membrane material can be inorganic, organic or mixtures thereof. Ceramic membranes are known, organic membranes are also known. Preferred materials for such membranes include polyamides, polybenzoimides, polysulfones (including sulfonated polysulfone and sulfonated polyethersulfones), polystyrenes including styrene containing random and blocked polymers, polycarbonates, cellulosic polymers such as cellulose acetate, cellulose acetate butyrate, polypropylene, polyvinyl chloride, polyethylene terephthalate, polyvinyl alcohol, fluorocarbons and other similar polymers that can obtain the porous structure needed for a molecular weight cut off. Such MWCO can be often formed on a porous support material in order to provide mechanical stability and integrity. Useful membranes include porous polysulfone manufactured by Minntech Corporation, Plymouth Minn.

The detection, analytic and monitoring system and methods of detection analysis and monitoring generally include a micro cantilever device positioned within the sensor having the detection system of the invention coated on the cantilever structure. Preferred molecular weight cut off membrane comprises a polysulfone membrane which can have a molecular weight cut off that ranges from about 10³ to about 10⁶.

### Sensor Placement

We have found that sensor placement requires that the sensor be placed subcutaneously but within fluid contact with or by a fluid that contains a glucose concentration indicative of or proportional to the concentration of glucose in venous blood. Such a location includes generally subcutaneously, in a vein, in the abdominal cavity or elsewhere where the sensor can come into contact with a representative fluid.

### Glucose Competition Binding Assay

Glucose competition was next assessed using a format more closely related to the format that will eventually be used in the final device. Previously selected diols that demonstrated a range of K_{eq} values with several of the DBAs relative to glucose were covalently immobilized on glass supports as iDIOL environments. A series of mixtures that contained a fixed concentration of fluorescentlylabeled DBA with varying concentrations of glucose, including the concentration range encompassing physiologically relevant glucose levels (30-300 mg/dL), were incubated with the iDIOL-functionalized surface. Detection of free, labeled DBA indicated loss of fluorescent signal from the iDIOL environment following exposure to glucose, confirming successful competition. A plot of the fluorescence signal in response to increasing glucose concentrations produced a response curve that defined the glucose sensitivity of the candidate DBA relative to the iDIOL. Response profiles of DBAs that showed a significant, competitive response to increasing glucose concentration were considered to have a desirable binding equilibrium between glucose and the iDIOL. On one hand, the DBA needed to bind to the iDIOL with sufficient affinity to produce a useful signal. On the other hand, the DBA needed to bind to the iDIOL weakly enough relative to the DBA:glucose affinity so that glucose could compete to produce a signal. The slope and IC₅₀ values of each response curve were the parameters used to compare the binding sensitivity of each DBA:glucose:iDIOL detection system.

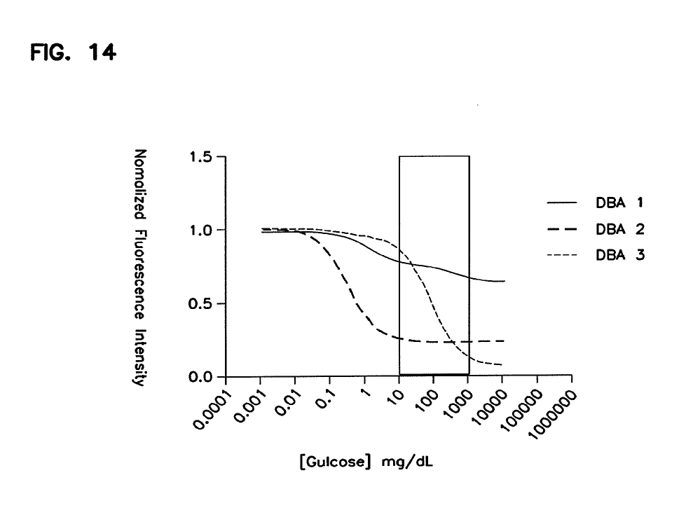

In one representative study, multiple candidate DBAs were used to generate glucose response curves using a reference iDIOL, over a broad glucose concentration range. FIG. 14 shows the glucose response curves, which are the inverse of the free solution fluorescence intensity measured during the assay. Upon addition of glucose, the fluorescence intensity of DBA not bound to the iDIOL increased. This was due to the competitive binding of glucose to the boronic acid receptors of the DBA, which prevented the fluorescentlylabeled DBA from binding to the iDIOL.

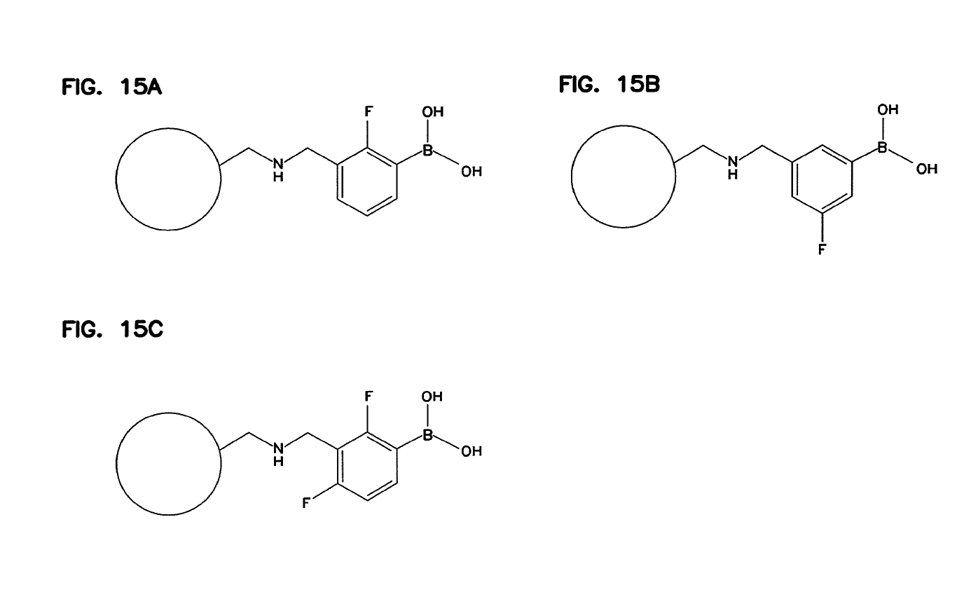

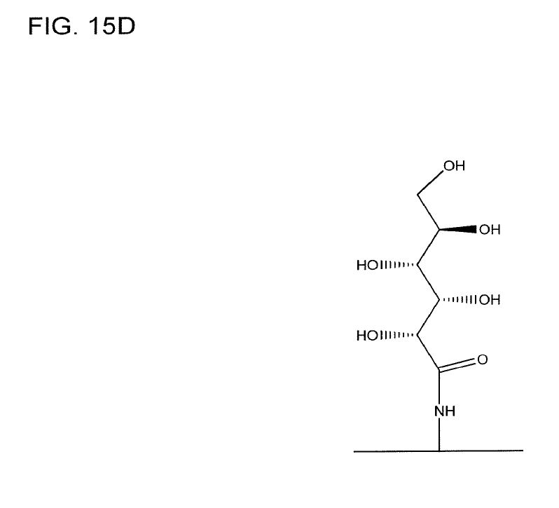

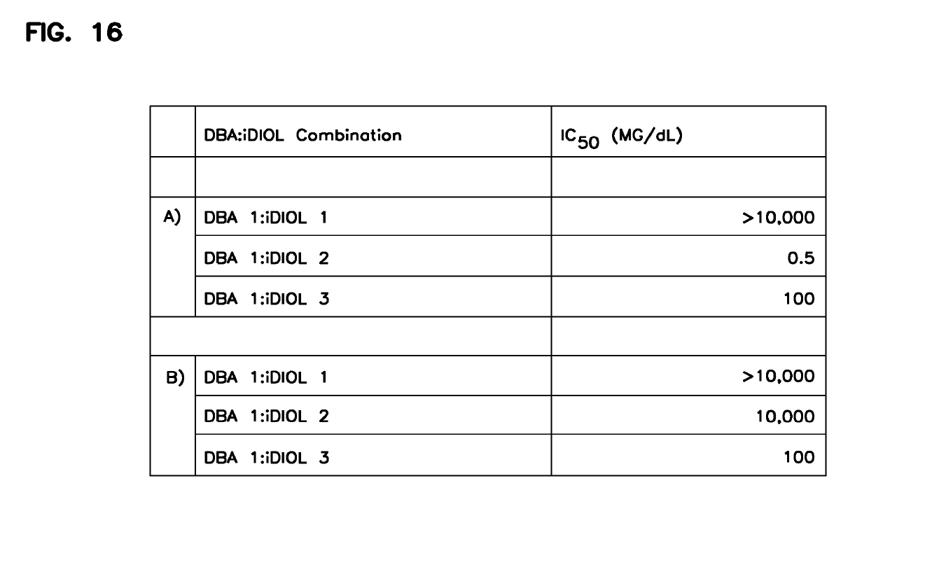

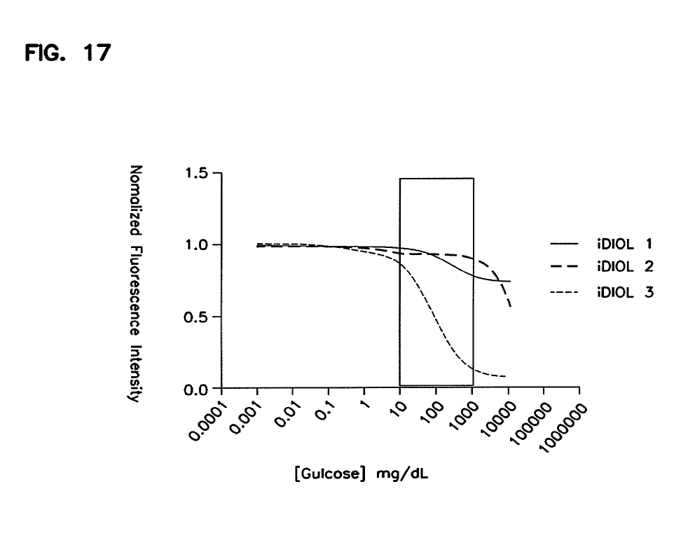

The candidate DBAs (FIG. 15A-C) respond differently to changing levels of glucose when exposed to a particular iDIOL (FIG. 15D), as would be expected from their DBA:diol K_{eq} values. DBA 2 and DBA 3 are on or below the DBA:glucose versus DBA:diol 1:1 line, indicating that glucose has equal or greater affinity for DBA 2 and DBA 3 than the diol. The opposite is true for DBA 1, which has minimal DBA:glucose affinity relative to the DBA:iDIOL. These data correlate with the observed glucose response curves where DBA 1 produced a minimally responsive curve and DBA 2 and DBA 3 showed typical competitive assay curves. Furthermore, the greater IC₅₀ sensitivity of DBA 2 relative to DBA 3 (FIG. 16) is in agreement with the difference in DBA 2:glucose binding affinity versus DBA 3:glucose binding affinity.

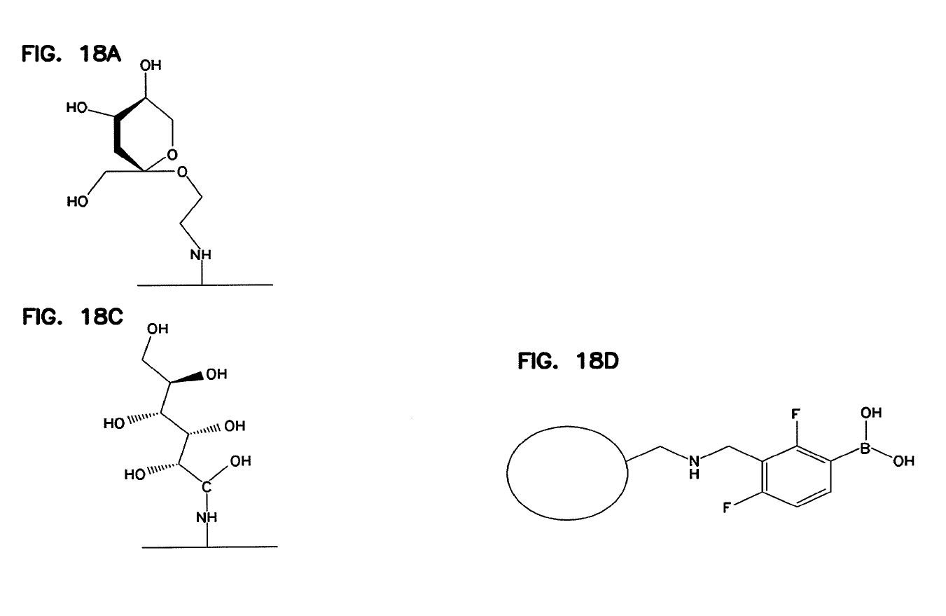

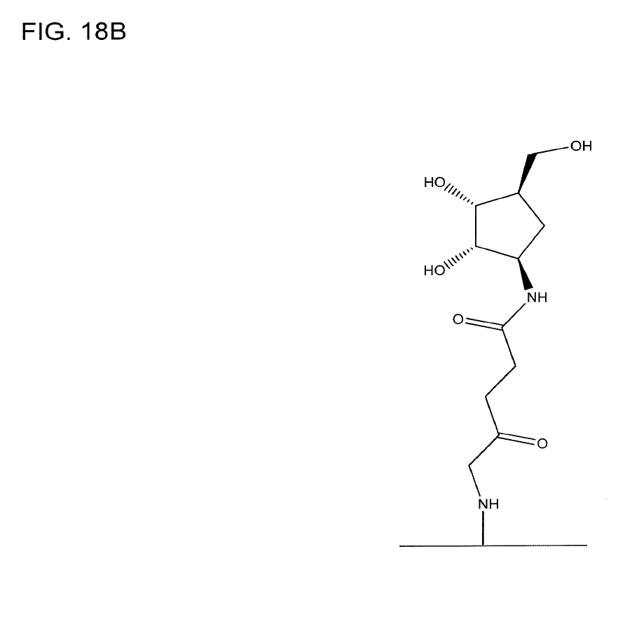

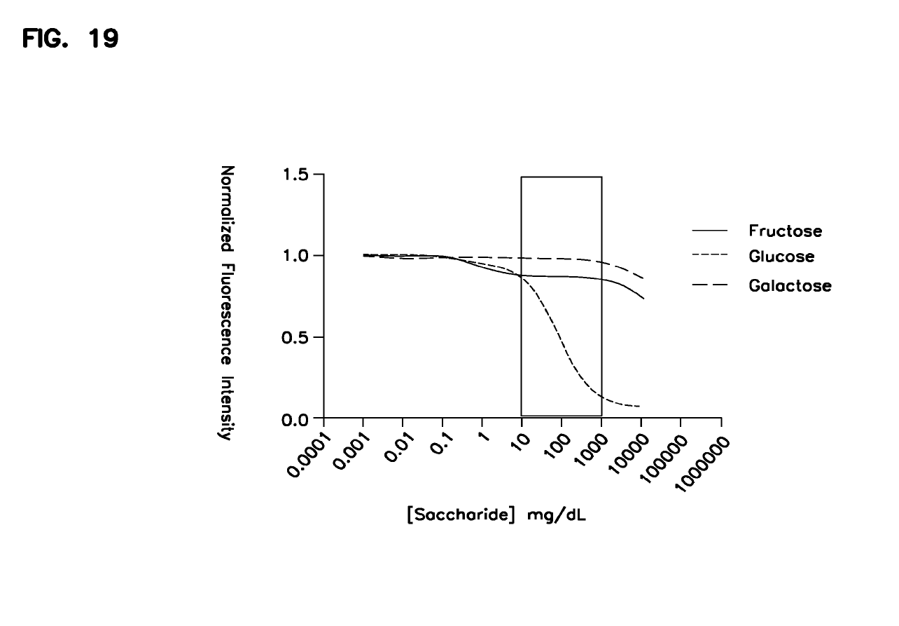

In a second representative study, multiple candidate iDIOL conjugates (FIG. 18A-C) were used to generate glucose response curves (FIG. 17) using a reference DBA (FIG. 18D) over a broad glucose concentration range. As in the previous example, upon addition of glucose, the fluorescence intensity of unbound DBA increased due to the competitive binding of glucose to the boronic acid receptors of the DBA, which prevented further binding of the DBA to the iDIOL surface. These glucose competition curves illustrate that the DBA responded, as would be expected from their DBA:diol K_{eq} values, to changing levels of glucose with significant diversity relative to the iDIOLs. Previously determined binding constants for DBA:glucose and DBA:diol combinations were correlated with the glucose response curves of each DBA:iDIOL system (FIG. 17). K_{eq} values for the diols corresponding to diol 1 and diol 2 are above the DBA:glucose versus DBA:diol 1:1 line, indicating that the dBA has less affinity for glucose than either diol, that correspond to iDIOL 1 and iDIOL 2. The opposite is true for the diol that corresponds to iDIOL 3, which lies below the 1:1 line. These data correlate with the observed glucose response curves, wherein iDIOL 1 and iDIOL 2 produce minimally responsive curves while iDIOL 3 produced a competitive assay curve.

Although the above studies established the glucose sensitivity of the illustrated DBA:iDIOL systems, it was also critical to determine glucose specificity. In a representative selectivity study, the DBA 3:iDIOL 3 component pair was evaluated for binding response relative to fructose and galactose (FIG. 19), which are present in vivo and could potentially interfere with the glucose response of the system. Measurements were performed over a broad saccharide concentration range. Upon addition of fructose and/or galactose, the DBA fluorescence intensity signal changed very little due to the inability of fructose and/or galactose to bind to the boronic acid receptors of the DBA. Therefore, the binding equilibrium of the DBA with the iDIOL binding environment was undisturbed. These curves show that this DBA:iDIOL pair is minimally cross-reactive with fructose or galactose.

Through these experiments, a selective glucose competition assay was established based on the binding affinities of DBAs for glucose and for an iDIOL surface. Additionally, our studies confirmed that candidate DBA:iDIOL pairs can be successfully screened for glucose sensitivity and selectivity. We have demonstrated that it is possible to use K_{eq} values to compare the binding affinities of a DBA for glucose and of the same DBA for an iDIOL. This enabled us to qualitatively predict the glucose-competitive response of each DBA:iDIOL pair and to select candidate pairs that will generate reproducible glucose response curves with optimal sensitivity and selectivity. Intuitively, it can be assumed that component pairs that fall on either extreme of the K_{eq} interaction graph will generate undesirable glucose response curves. On one end of the DBA:iDIOL affinity spectrum, the DBA binds too strongly to the iDIOL and glucose cannot effectively compete. On the other end of the affinity spectrum, the DBA binds too weakly to the iDIOL, which will not provide a useful dynamic range. With the capability of predicting glucose response curves based on the location of a K_{eq} data interaction point, it was possible for us to quickly and efficiently eliminate component pair combinations that would be expected to perform in subsequent studies with low sensitivity and selectivity. Much to our advantage, this screening approach drastically limits the number of experiments that are required to select the best DBA:iDIOL combination, reducing time and cost investments. The results discussed above establish the validity of the K_{eq} data interaction model for selection of candidate DBA:iDIOL pairs. The diversity of responses generated by each DBA:glucose:iDIOL system within our library ensures that we will be able to select DBA:iDIOL pairs with the appropriate physical and chemical properties necessary for analyzing glucose concentrations within the sensitivity and selectivity parameters required by the final device.

### EXPERIMENTAL

In the following experimental work we have taken selected DBA structures and labeled those structures with a fluorescent dye and used those structures with an polyol immobilized on a glass slide/platform surface. We have demonstrated that we can efficiently immobilize the polyol on a glass slide/platform surface, synthesize the appropriate DBA and show that the iDIOL: DBA system can be used in analyte detection or quantification. We have used this test set up to demonstrate that we can determine or quantify K_{ad} and K_{id} of materials of the system and that the system can provide a quantitative glucose determination. We believe the demonstration of a quantitative glucose analysis shows that the system can be generalized to other analyte analyses.

Ethylenediamine-core poly(amidoamine) (PAMAM) generation 1 [G1] dendrimer and generation 2 [G2] dendrimer containing eight amine surface functional groups (47.92% (w/w) in methanol) and sixteen amine surface functional groups (31.83% (w/w) in methanol), respectively, were purchased from Dendritech. Aryl boronic acids were purchased from Combi-Blocks. D-(+)-Glucose, D-(−)-Fructose, D-(+)-Galactose, gluconolactone, N-(3-Dimethylaminopropyl)-N-ethylcarbodiimide hydrochloride (EDC), sodium borohydride (NaBH₄), 4-dimethylaminopyridine (DMAP), succinic anhydride, glucose oxidase, from Aspergillus niger, 1× phosphate buffer saline (1×PBS), anhydrous methanol (MeOH), N,N-dimethylacetamide (DMA), N,N-dimethylformamide, CHROMASOLV®, HPLC grade water were purchased from Sigma-Aldrich. N-Hydroxysulfosuccinimide (Sulfo-NHS) was purchased from Thermo Scientific. Alexa Fluor® 647 carboxylic acid (fluorescent dye), succinimidyl ester was purchased from Invitrogen. All chemicals were used as received. UltraGAPS® amine coated glass slides were purchased from Corning Life Sciences. Bio-Gel, P-2 size exclusion chromatography resin was purchased from BioRad. Amine functionalized controlled pore glass chromatography media (1000 Å) was purchased from Millipore.

### Example 1A-1H

### Ex. 1

### Synthesis of Gluconolactone Polyol Immobilized on Class Surface

First, glass slides were functionalized. Amine-functionalized glass slides were fully immersed in a lid tight Coplin jar containing a 0.5 M solution of gluconolactone dissolved in buffer (85% DMA, 15% HPLC grade water containing 1 mg mL⁻¹ DMAP). The slides were incubated at 25° C. overnight and then washed with water. Unreacted amines were blocked by immersing the slides in a solution containing 0.1% succinic anhydride in DMF and allowing them to incubate at 25° C. overnight, followed by a DMF and then water wash.

### Ex. 1A

### Gluconolactone Polyol Immobilization on Amine Functionalized Controlled Pore Glass

To 0.5 g of CPG-NH₂ (1000 Å, Millipore), add 3 mL of a 0.7 M solution of gluconolactone dissolved in a mixture containing 85% DMA, 15% HPLC grade water and 1 mg mL⁻¹ DMAP. Mix overnight on orbital shaker (170 rpm) at 25° C. Isolate modified CPG using vacuum filtration followed by three DMA and then three water washes. Unreacted amines were blocked by adding 3 mL of a 1 M solution of succinic anhydride in DMF to the 0.5 g batch of modified CPG. Mix overnight on orbital shaker (170 rpm) at room 25°. Isolate modified CPG using vacuum filtration followed by three DMF and then three water washes.

### Ex 1B

### (1S,2R,3S,4S)-2,3-Dihydroxy-4-(hydroxymethyl)-1-aminocyclopentane hydrochloride Polyol Immobilization on Amine Functionalized Controlled Pore Glass

To 0.5 g of CPG-NH2 (1000 Å, Millipore), add 3 mL of a 1 M solution of succinic anhydride in DMF. Mix overnight on orbital shaker (170 rpm) at 25° C. The modified CPG was isolated using vacuum filtration followed by three DMF and then three water washes and then allowed to dry. The carboxylic acid functionalized CPG was activated for 1 hour at 25° C. with a freshly prepared aqueous solution of N-(3-Dimethylaminopropyl)-N′-ethylcarbodiimide hydrochloride (EDC) (0.16 M)/N-hydroxysulfosuccinimide (Sulfo-NHS) (0.21 M) that was combined with DMF to make a 4:1 DMF:water mixture. Following activation, the CPG was isolated using vacuum filtration followed by three water washes and allowed to dry. The activated CPG was suspended in a 100 mM solution of (1S,2R,3S,4S)-2,3-Dihydroxy-4-(hydroxymethyl)-1-aminocyclopentane hydrochloride dissolved in 0.1 M sodium bicarbonate buffer (pH 8.5) and allowed to mix overnight on orbital shaker (170 rpm) at 25°. The modified CPG was isolated using vacuum filtration followed by three water washes and then allowed to dry.

### Ex. 1C

### 3-Chloro-1,2-propane Polyol Immobilization on Amine Functionalized Controlled Pore Glass

To 0.5 g of CPG-NH2 (1000 Å, Millipore), add 3 mL of a 1 M solution of succinic anhydride in DMF. Mix overnight on orbital shaker (170 rpm) at 25° C. The modified CPG was isolated using vacuum filtration followed by three DMF and then three water washes and then allowed to dry. The carboxylic acid functionalized CPG was suspended in a 100 mM solution of 3-Chloro-1,2-proopanediol in DMSO and allowed to mix overnight on orbital shaker (170 rpm) at 25° C. The modified CPG was isolated using vacuum filtration followed by three DMSO and then three water washes and then allowed to dry.

### Ex. 1D

### Valiolamine Hydrate Polyol Immobilization on Amine Functionalized Controlled Pore Glass

To 0.5 g of CPG-NH2 (1000 Å, Millipore), add 3 mL of a 1 M solution of succinic anhydride in DMF. Mix overnight on orbital shaker (170 rpm) at 25° C. The modified CPG was isolated using vacuum filtration followed by three DMF and then three water washes and then allowed to dry. The carboxylic acid functionalized CPG was activated for 1 hour at 25° C. with a freshly prepared aqueous solution of N-(3-Dimethylaminopropyl)-N′-ethylcarbodiimide hydrochloride (EDC) (0.16 M)/N-hydroxysulfosuccinimide (Sulfo-NHS) (0.21 M) that was combined with DMF to make a 4:1 DMF:water mixture. Following activation, the CPG was isolated using vacuum filtration followed by three water washes and allowed to dry. The activated CPG was suspended in a 100 mM solution of Valiolamine Hydrate dissolved in 0.1 M sodium bicarbonate buffer (pH 8.5) and allowed to mix overnight on orbital shaker (170 rpm) at 25° C. The modified CPG was isolated using vacuum filtration followed by three water washes and then allowed to dry.

### Ex. 1E

### 2-Chloroethyl-b-D-fructopyranoside Polyol Immobilization on Amine Functionalized Controlled Pore Glass

To 0.5 g of CPG-NH2 (1000 Å, Millipore), add 3 mL of a 100 mM solution of 2-Chloroethyl-b-D-fructopyranoside (Carbosynth Ltd.) in DMSO that contains N,N-Diisopropylethylamine (100 mM). Mix overnight on orbital shaker (170 rpm) at 25° C. The modified CPG was isolated using vacuum filtration followed by three DMSO and then three water washes and then allowed to dry.

### Ex. 1F

### (−)-2,3:4,6-Di-O-Isopropylidene-2-keto-L-gulonic acid monohydrate Polyol Immobilization (and Deprotection) on Amine Functionalized Controlled Pore Glass

1.2 mL of a 100 mM solution of (−)-2,3:4,6-Di-O-Isopropylidene-2-keto-L-gulonic acid monohydrate in DMF was activated for 1 hour at 25° C. with 0.3 mL of a freshly prepared aqueous solution of N-(3-Dimethylaminopropyl)-N′-ethylcarbodiimide hydrochloride (EDC) (0.64 M)/N-hydroxysulfosuccinimide (Sulfo-NHS) (0.14 M). The activated acid was diluted with 1.5 mL of 0.1 M sodium bicarbonate buffer (pH 8.5) and added to 0.5 g of CPG-NH2 (1000 Å, Millipore) and then allowed to mix overnight on orbital shaker (170 rpm) at 25° C. The modified CPG was isolated using vacuum filtration followed by three DMF washes and then three water washes and then allowed to dry. The modified CPG was resuspended in a 9:1 mixture of trifluoroacetic acid (TFA):water and allowed to mix overnight on orbital shaker (170 rpm) at 25° C. The modified CPG was isolated using vacuum filtration followed by three water washes and then allowed to dry.

### Ex. 1G

### (1S,2R,3S,4S)-2,3-Dihydroxy-4-(hydroxymethyl)-1-aminocyclopentane hydrochloride Polyol Immobilization on Glass Slides

An amine-functionalized glass slide was fully immersed in a lid tight Coplin jar containing a 1 M solution of succinic anhydride dissolved in DMF. The slide was incubated at 25° C. overnight and then washed with DMF and then water and centrifuged to dry. The carboxylic acid functionalized glass slide was activated for 1 hour at 25° C. with a freshly prepared aqueous solution of N-(3-Dimethylaminopropyl)-N′-ethylcarbodiimide hydrochloride (EDC) (0.16 M)/N-hydroxysulfosuccinimide (Sulfo-NHS) (0.21 M) that was combined with DMF to make a 4:1 DMF:water mixture. Following activation, the slide was rinsed with water and then centrifuged to dry. The activated slide was immersed in a 100 mM solution of (1S,2R, 3S, 4S)-2,3-Dihydroxy-4-(hydroxymethyl)-1-aminocyclopentane hydrochloride dissolved in 0.1 M sodium bicarbonate buffer (pH 8.5) and allowed to incubate overnight in a humid chamber at 25° C. The slide was rinsed with water and then centrifuged to dry.

### Ex. 1H

### 3-Chloro-1,2-propane Polyol Immobilization on Glass Slides

An amine-functionalized glass slide was fully immersed in a lid tight Coplin jar containing a 1 M solution of succinic anhydride dissolved in DMF. The slide was incubated at 25° C. overnight and then was rinsed with DMF and then water and centrifuged to dry. The slide was immersed in a 100 mM solution of 3-Chloro-1,2-propaneiDIOL dissolved in DMSO and allowed to incubate overnight in a humid chamber at 25° C. The slide was rinsed with DMSO and then water and centrifuged to dry.

### Ex. 1I

### Valiolamine Hydrate Polyol Immobilization on Glass Slides

An amine-functionalized glass slide was fully immersed in a lid tight Coplin jar containing 1 M solution of succinic anhydride dissolved in DMF. The slide was incubated at 25° C. overnight and then was rinsed with DMF and then water and centrifuged to dry. The carboxylic acid functionalized glass slide was activated for 1 hour at 25° C. with a freshly prepared aqueous solution of EDC (0.16 M)/Sulfo-NHS (0.21 M) that was combined with DMF to make a 4:1 DMF:water mixture. Following activation, the slide was rinsed with water and then centrifuged to dry. The activated slide was immersed in a 100 mM solution of Valiolamine Hydrate dissolved in 0.1 M sodium bicarbonate buffer (pH 8.5) and allowed to incubate overnight in a humid chamber at 25° C. The slide was rinsed with water and then centrifuged to dry.

### Ex1J

### 2-Chloroethyl-b-D-fructopyranoside Polyol Immobilization on Glass Slides

An amine-functionalized glass slide was fully immersed in a 100 mM solution of 2-Chloroethyl-b-D-fructopyranoside (Carbosynth Ltd) in DMSO that contains N,N-Di-isopropylethylamine (100 mM). The slide was incubated at 25° C. overnight and then rinsed with DMSO and then water and centrifuged to dry.

### Ex 1H

### (−)-2,3:4,6-Di-O-isopropylidene-2-keto-L-gulonic acid monohydrate Polyol Immobilization (and Deprotection) on Glass Slides

(−)-2,3:4,6-Di-O-isopropylidene-2-keto-L-gulonic acid monohydrate was dissolved in DMF (100 mM) and then activated for 1 hour at 25° C. with a freshly prepared aqueous solution of N-(3-Dimethylaminopropyl)-N′-ethylcarbodiimide hydrochloride (EDC) (0.64 M)/N-hydroxysulfosuccinimide (Sulfo-NHS) (0.14 M). The activated acid was diluted (1:1) with 0.1 M sodium bicarbonate buffer (pH 8.5). An amine-functionalized slide was fully immersed in the 1:1 solution and incubated at 25° C. overnight and then rinsed with DMF and then water and centrifuged to dry. The modified slide was fully immersed in a 9:1 mixture of trifluoroacetic acid (TFA):water and allowed to incubate at 25° C. overnight and then rinsed with water and centrifuged to dry.

### Examples 2-12

### Synthesis of PAMAM Generation 1 Dendrimers Boronic Acid [G1]-1[G1]-12

In separate reactions, to a solution of generation 1, ethylenediamine-core PAMAM dendrimer (500 mg, 0.35 mmol) in anhydrous MeOH (25 mL) was added 16-fold molar excess of each boronic acid (1, 2, 3, 4, 6, 7, 8, 9, 10, 11, 12 see Table 1). Each solution was stirred for 48 h at 60° C. under a positive pressure of argon in a appropriately sized round bottom flask. The reaction mixtures were then cooled to 0° C. using an ice bath in water after which NaBH₄ (212 mg, 5.59 mmol) was added in portions under a flow of argon. The contents of each reaction were brought to room temperature and allowed to further stir overnight. Two molar HCl (aq) was added drop-wise until the formation of gas ceased and the solution allowed to stir for 2 h. The crude contents were neutralized with NaOH (aq) and diluted with 12.5 mL MeOH and 12.5 mL water mixture and then purified by passing through a ultra filtration membrane (MWCO 1000) at 60 psi argon pressure in a Millipore stirred cell. The product was further isolated with 2×12.5 mL of 50% MeOH (aq) using the same cell. Purified material was retrieved by dissolving in MeOH and evaporated (Rotovap®) to give a pale yellow, translucent gum with a yield of 84% (421 mg).

### Examples 13-24

### Synthesis of PAMAM Generation 2 Boronic Acid Dendrimers [G2]-15-[G2]-26

Similar to examples 2-11, separably, to a solution of generation 2, ethylenediamine-core PAMAM dendrimer (500 mg, 0.15 mmol) in anhydrous MeOH (25 mL) was added 32-fold molar excess of boronic acid (1, 2, 3, 4, 5, 6, 7, 8, 9, 10, 11, 12 or table 1). Each solution was stirred for 48 h at 60° C. under a positive pressure of argon. The reaction mixtures were then cooled to 0° C. using an ice bath in water after which NaBH₄ (186 mg, 4.91 mmol) was added in portions under a flow of argon. The contents of each reaction were brought to room temperature and allowed to further stir overnight. 2 M HCl (aq) was added drop-wise until the formation of gas ceased and the solution allowed to stir for 2 hours. The crude contents were neutralized with NaOH (aq) and diluted with 12.5 mL MeOH and 12.5 mL water mixture and then purified by passing through a ultrafiltration membrane (MWCO 3000) at 60 psi argon pressure in a Millipore stirred cell. The product was further isolated with 2×12.5 mL of 50% MeOH (aq) using the same cell. Purified material was retrieved by dissolving in MeOH and evaporated (Rotovap®) to give a pale yellow, translucent gum with a yield of 85% (427 mg).

### Examples 22A-22D

### Ex. 22A

### Carboxyl Boronic Acid—PAMAM DBA Synthesis

Similar to Examples 2-11, 4-Borono-2-(trifluoromethyl)benzoic acid (0.26 g, 1.12 mmol), dissolved in DMF (0.8 mL), was activated for 1 hour at 25° C. with 0.2 mL of a freshly prepared aqueous solution of N-(3-Dimethylaminopropyl)-N′-ethylcarbodiimide hydrochloride (EDC) (0.32 g, 1.68 mmol)/N-hydroxysulfosuccinimide (Sulfo-NHS) (0.47 g, 2.24 mmol). The activated acid was added to a solution of generation 1, ethylenediamine-core PAMAM dendrimer (100 mg, 0.07 mmol), dissolved in 4 mL of a 1:1 DMF:0.1 M sodium bicarbonate buffer (pH 8.5) mixture and stirred for 24 hours in a appropriately sized round bottom flask. The volume of the reaction was reduced to dryness and redissolved in 20 mM ammonium acetate buffer. The crude contents were then purified by size exclusion chromatography using P2 Biogel (BioRad) matrix packed in a polypropylene Econo-Pac Column (1.5×12 cm, 20 mL total volume, BioRad). The column was equilibrated with 20 mM ammonium acetate buffer and run by gravity flow. Purified material was retrieved by evaporating buffer using a Savant SpeedVac Concentrator (ThermoFisher) to give a pale yellow, translucent gum with a yield of 65% (65 mg).

### Examples 22B

Generation 1: 3-Carboxy-5-nitrophenylboronic acid (0.24 g, 1.12 mmol) is used.

### Examples 22C

Generation 2 (100 mg, 0.031 mmol): 4-Borono-2-(trifluoromethyl)benzoic acid (0.23 g, 0.98 mmol) 3-Carboxy-5-nitrophenylboronic acid (0.21 g, 0.98 mmol) EDC (0.28 g, 1.47 mmol) Sulfo-NHS (0.43 g, 1.97 mmol) is used.

### Examples 22D

Generation 3 (100 mg, 0.014 mmol): 4-Borono-2-(trifluoromethyl)benzoic acid (0.22 g, 0.93 mmol) 3-Carboxy-5-nitrophenylboronic acid (0.19 g, 0.93 mmol) EDC (0.26 g, 1.39 mmol) Sulfo-NHS (0.40, 1.85 mmol) is used.

### Examples 22E

Generation 4 (100 mg, 0.0070 mmol): 4-Borono-2-(trifluoromethyl)benzoic acid (0.21 g, 0.90 mmol) 3-Carboxy-5-nitrophenylboronic acid (0.19 g, 0.90 mmol) EDC (0.26 g, 1.35 mmol) Sulfo-NHS (0.39 g, 1.8 mmol) is used.

### Preparation of Fluorescently Labeled Boronic Acid Dendrimers

### Examples 23-33

### Fluorophore (Alexa Fluor® 647)-PAMAM [G1]1-[G1]14 Boronic Acid Dendrimers

Each of generation 1 boronic acid dendrimers 1, 2, 3, 4, 5, 6, 7, 8, 9, 10, 11, 12, 13 or 14 (table 2) (2 mg, 0.0014 mmol) were dissolved in 100 uL of 0.1 M NaHCO₃ buffer, pH 8.3. A 100 uL solution of a fluorescent compound, Alexa Fluor® carboxylic acid, succinimidyl ester, in anhydrous DMSO (10 mg mL⁻¹, 0.0014 mmol) was added to each and allowed to stir overnight in a screw capped vial. The crude materials in each reaction vessel were passed through size exclusion resin (Bio-Gel, P-2 Gel) and the remaining residue was retrieved by centrifugal evaporation with a yield of 90% (1.8 mg).

### Examples 34-45

### Fluorophore (Alexa Fluor® 647)-PAMAM [G2]15-[G2]28 Boronic Acid Dendrimers

Generation 2 boronic acid dendrimers 15, 16, 17, 18, 19, 20, 21, 22, 23, 24, 25, 26, 27, or 28 (table 2) (2 mg, 0.0006 mmol) were dissolved in 100 μL of 0.1 M NaHCO₃ buffer, pH 8.3. A 40 μL solution of Alexa Fluor® carboxylic acid, succinimidyl ester in anhydrous DMSO (10 mg mL⁻¹, 0.0006 mmol) was added and allowed to stir overnight. The crude material was passed through size exclusion resin (Bio-Gel, P-2 Gel) and the remaining residue was retrieved by centrifugal evaporation with a yield of 95% (1.9 mg).

### Examples 51-64

### Fluorophore (Alexa Fluor® 647) [G3]29-[G3]42 Boronic Acid Dendrimers

Generation 3 boronic acid dendrimers 29, 30, 31, 32, 33, 34, 35, 36, 37, 38, 39, 40, 41, or 42 (See Table 2) (2 mg, 0.0003 mmol) were dissolved in 100 uL of 0.1 M NaHCO₃ buffer, pH 8.3. A 40 uL solution of Alexa Fluor® carboxlic acid succinimidyl ester in anhydrous DMSO (10 mg mL⁻¹, 0.0003 mmol) was added and allowed to stir overnight. The crude material was passed through size exclusion resin (Bio-Gel, P-2 Gel) and the remaining residue was retrieved by centrifugal evaporation with a yield of 72% (1.44 mg).

### Examples 65-78

### Fluorophore (Alexa Fluor® 647) [G4]43-[G4]56 Boronic Acid Dendrimers

Generation 4 boronic acid dendrimers 43, 44, 45, 46, 47, 48, 49, 50, 51, 52, 53, 54, 55, or 56 (See Table 2) (2 mg, 0.0001 mmol) were dissolved in 100 uL of 0.1 M NaHCO₃ buffer, pH 8.3. A 40 uL solution of Alexa Fluor® carboxlic acid succinimidyl ester in anhydrous DMSO (10 mg mL⁻¹, 0.0003 mmol) was added and allowed to stir overnight. The crude material was passed through size exclusion resin (Bio-Gel, P-2 Gel) and the remaining residue was retrieved by centrifugal evaporation with a yield of 48% (0.96 mg).

The materials from Examples 1-45 were used in experiments to first establish workable K_{gd} and K_{id} for a glucose analysis and then demonstrate that a quantitative test can be obtained.

### Examples 79A-79L

### Synthesis of PAMAM Generation 3 Boronic Acid Dendrimers [G3]-29-[G3]-40

Similar to examples 2-11, separably, to a solution of generation 3, ethylenediamine-core PAMAM dendrimer (500 mg, 0.07 mmol) in anhydrous MeOH (25 mL) was added 64-fold molar excess of boronic acid (1, 2, 3, 4, 5, 6, 7, 8, 9, 10, 11, 12 or table 1). Each solution was stirred for 48 h at 60° C. under a positive pressure of argon. The reaction mixtures were then cooled to 0° C. using an ice bath in water after which NaBH₄ (175 mg, 4.63 mmol) was added in portions under a flow of argon. The contents of each reaction were brought to room temperature and allowed to further stir overnight. 2 M HCl (aq) was added drop-wise until the formation of gas ceased and the solution allowed to stir for 2 hours. The crude contents were neutralized with NaOH (aq) and diluted with 12.5 mL MeOH and 12.5 mL water mixture and then purified by passing through a ultrafiltration membrane (MWCO 3000) at 60 psi argon pressure in a Millipore stirred cell. The product was further isolated with 2×12.5 mL of 50% MeOH (aq) using the same cell. Purified material was retrieved by dissolving in MeOH and evaporated (Rotovap®) to give a pale yellow, translucent gum with a yield of 65% (325 mg).

### Examples 80A-80L

### Synthesis of PAMAM Generation 4 Boronic Acid Dendrimers [G4]-43-[G4]-54

Similar to examples 2-11, separably, to a solution of generation 4, ethylenediamine-core PAMAM dendrimer (500 mg, 0.04 mmol) in anhydrous MeOH (25 mL) was added 128-fold molar excess of boronic acid (1, 2, 3, 4, 5, 6, 7, 8, 9, 10, 11, 12 or table 1). Each solution was stirred for 48 h at 60° C. under a positive pressure of argon. The reaction mixtures were then cooled to 0° C. using an ice bath in water after which NaBH₄ (170 mg, 4.49 mmol) was added in portions under a flow of argon. The contents of each reaction were brought to room temperature and allowed to further stir overnight. 2 M HCl (aq) was added drop-wise until the formation of gas ceased and the solution allowed to stir for 2 hours. The crude contents were neutralized with NaOH (aq) and diluted with 12.5 mL MeOH and 12.5 mL water mixture and then purified by passing through a ultrafiltration membrane (MWCO 3000) at 60 psi argon pressure in a Millipore stirred cell. The product was further isolated with 2×12.5 mL of 50% MeOH (aq) using the same cell. Purified material was retrieved by dissolving in MeOH and evaporated (Rotovap®) to give a pale yellow, translucent gum with a yield of 48% (240 mg).

### Glucose Competition Assay in 1×PBS

A 48 nM (by mass) Alexa Fluor® boronic acid dendrimer solution was prepared in 1× phosphate buffered saline (PBS). A concentration dilution series of D-(+)-Glucose solutions, which included a range from 10,000,000× (0.48 M) to 0× (0 M) the mass of the boronic acid dendrimer, were prepared in 1×PBS. 2 μL of a working solution containing 1 part 0.48 M D-(+)-Glucose and 1 part 48 nM Alexa Fluor® boronic acid dendrimer were spotted (in triplicate) on a gluconolactone immobilized glass slide. This was repeated for each D-(+)-Glucose concentration working solution. The slide was allowed to incubate for 1 h in a humid chamber after which it was washed with 1×PBS. While those use a fluorescent detection other detection systems can be used.

### Glucose Competition in Matrix

A glucose free plasma matrix was made by taking a 2 mL volume of fractionated plasma that was separated from the buffy coat and erythrocyte layer of a whole blood sample and treated with glucose oxidase for 1 h. A stock solution of matrix was created by dialyzing the glucose free plasma matrix through a 10 k cut-off dialysis membrane into 20 mL of 1×PBS overnight at 4° C.

A 48 nM (by mass) Alexa Fluor® boronic acid dendrimer solution was prepared in matrix. A concentration dilution series of D-(+)-Glucose solutions, which included a range from 10,000,000× (0.48 M) to 0× (0 M) the mass of the boronic acid dendrimer, were prepared in matrix. 2 μL of a working solution containing 1 part 0.48 M D-(+)-Glucose and 1 part 48 nM Alexa Fluor® boronic acid dendrimer were spotted (in triplicate) on a gluconolactone immobilized glass slide. This was repeated for each D-(+)-Glucose concentration working solution. The slide was allowed to incubate for 1 h in a humid chamber after which was washed with 1×PBS.

### Imaging and Data Analysis

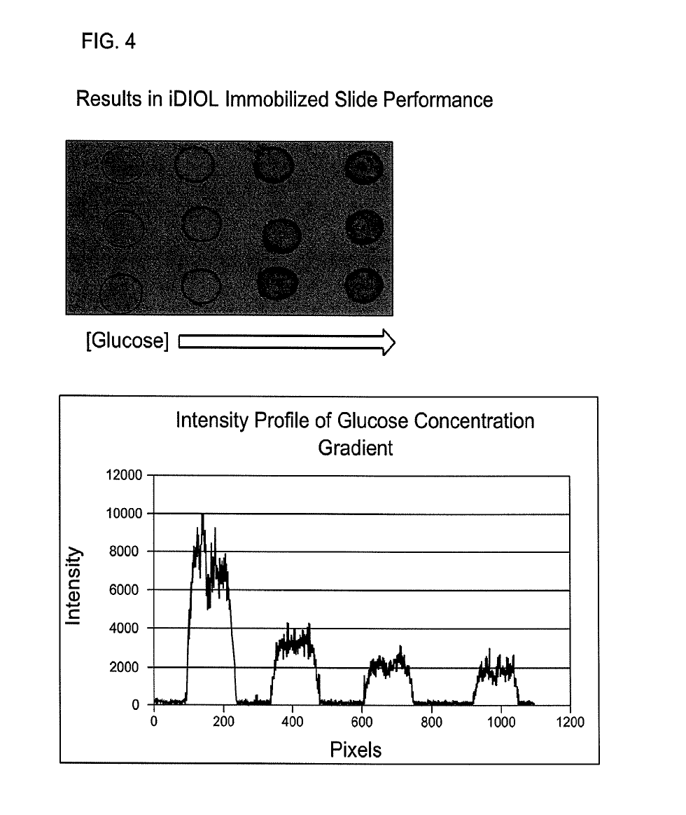

Slides were scanned with a 635 nm laser using a GenePix Personal 4100A Microarray Scanner (Axon Instruments, Union City, Calif.). Analysis was done with the software package, Acuity® 4.0 Microarray Informatics Software. The fluorescent signals were analyzed by quantifying the mean pixel density or intensity of each 2 μL spot area (μm²) and then using that data for analysis. Glucose competition curves were generated by plotting the concentration of D-(+)-Glucose vs. the average relative fluorescent units (RFU) for each working solution. FIG. 4 shows a photographic representation and a graphical representation of the intensity profile of glucose concentration gradients.

### Glucose Competition Assay II

Alizarin Red S. (ARS), and a saccharide that can be immobilized on the surface, commercially available saccharides (diols) and buffer materials were purchased from Sigma-Aldrich, Acros, and Carbosynth, Ltd. Custom synthesized saccharides were purchased from Gateway Chemical Technology, Inc. Dendrimer-Boronic Acids (DBAs) were prepared as described in Tables 1 and 2.

Determination of K_{id} (binding constant) for each (DBA)-(diol) equilibrium was based off a previously established literature method. A three component competitive assay containing ARS, a DBA and an diol was used to examine the competing equilibrium of each of the components of this specific system, the first being the association constant, a K_{id}, between each DBA and ARS and the second being the K_{eq} association constant between each DBA and each diol (K_{ad}).

### Binding Affinity (K_{id}) Calculation of ARS-DBA Complex

A series of DBA concentrations (10-200 equivalents) were prepared in a solution of ARS (9.0×10⁻⁶ M) in a 1× phosphate buffered saline solution (1×PBS). The relative fluorescent intensities were measured using an excitation wavelength of 468 nm and an emission wavelength of 572 nm. K_{id} is the quotient of the intercept and the slope of the plot 1/[DBA] vs. 1/ΔF.

### Binding Affinity (K_{ad}) Calculation of Saccharide-DBA Complex

A concentration of DBA (2.0×10⁻³ M) was prepared in a solution of ARS (9.0×10⁻⁶ M) in 1×PBS. The iDIOL, 1, 2, 3, 4, 5, 6, 7, 8, 9, 10, 11, 12, 13, 14, 15, 16 or 17 (FIG. 6) was added to the DBA-ARS solution with a range of concentrations from 2 M down to 9.76×10⁻⁶ M. The relative fluorescent intensities were measured using an excitation wavelength of 468 nm and an emission wavelength of 572 nm. K_{eqd1} is the quotient of K_{ad} and the slope from plot 1/P vs. Q where:

P=[Lₒ]−1/QK_{eqd1}−[Iₒ]/(Q+1)
Lₒ=total amount of dendrimer-boronic acid (DBA)
Iₒ=total amount ARS
K_{eqd1}=binding affinity of ARS-DBA complex
Q=change in fluorescence of the solution

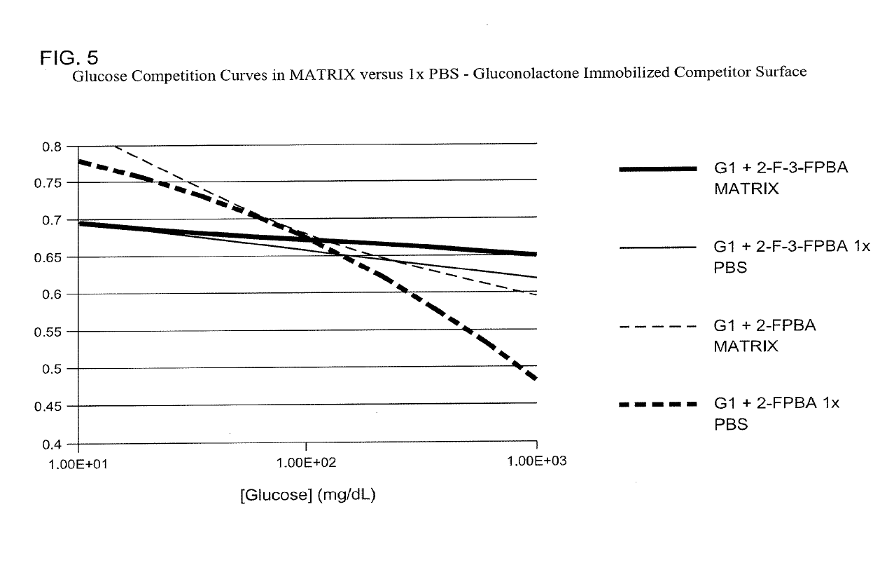

FIGS. 4 and 5 are representations of the results of quantitative analysis of glucose using the glucose, DBA, iDIOL system of the invention and shows sensitivity sufficient to obtain reliable glucose results.

Glucose Competition Assay Using iDIOL Modified CPG (in 2× Matrix, 5× Matrix, and 1×PBS) Examples of Synthesis of Polyol(s) Immobilized on Amine Derivatized Controlled Pore Glass (CPG) Media

Preparation of 2× Matrix—

A glucose free plasma matrix (2×) was made by taking a 300 mL volume of fractionated plasma that was separated from the buffy coat and erythrocyte layer of a whole blood sample and treated with glucose oxidase for 1 h. A stock solution of 2× matrix was created by dialyzing the glucose free plasma matrix through a 10 k cut-off dialysis membrane into 600 mL of 1×PBS overnight at 4° C. FIGS. 21A to 21D Show the data for the glucose competition in a plasma matrix with the noted materials.

Preparation of 5× Matrix—

A glucose free plasma matrix (5×) was made by taking a 300 mL volume of fractionated plasma that was separated from the buffy coat and erythrocyte layer of a whole blood sample and treated with glucose oxidase for 1 h. A stock solution of 5× matrix was created by dialyzing the glucose free plasma matrix through a 10 k cut-off dialysis membrane into 1500 mL of 1×PBS overnight at 4° C.

Glucose Competition Assay in 2× Matrix (or 5× Matrix or 1×PBS)

A 2688 nM (by mass) Alexa Fluor boronic acid dendrimer solution was prepared in 2× matrix (or 5× matrix or 1×PBS). A concentration dilution series of D-(+)-Glucose solutions, which included a range from 1,000,000×(2.688 M) to 0× (0 M) the mass of the boronic acid dendrimer, were prepared in 2× matrix (or 5× matrix or 1×PBS). 240 uL of a working solution containing 1 part 2.688 M glucose, 1 part 2688 nM Alexa Fluor boronic acid dendrimer and 2 parts 2× matrix (or 5× matrix or 1×PBS) was used to suspend 0.01 g of gluconolactone immobilized CPG in solution. The suspension of CPG in the working solution was continually mixed and incubated at 25° C. for 15 minutes. The CPG was allowed to settle and the supernatant was removed for analysis.

Fructose Competition Assay in 2× Matrix (or 5× Matrix or 1×PBS)

A 2688 nM (by mass) Alexa Fluor boronic acid dendrimer solution was prepared in 2× matrix (or 5× matrix or 1×PBS). A concentration dilution series of D-(−)-Fructose solutions, which included a range from 1,000,000×(2.688 M) to 0× (0 M) the mass of the boronic acid dendrimer, were prepared in 2× matrix (or 5× matrix or 1×PBS). 240 uL of a working solution containing 1 part 2.688 M fructose, 1 part 2688 nM Alexa Fluor boronic acid dendrimer and 2 parts 2× matrix (or 5× matrix or 1×PBS) was used to suspend 0.01 g of gluconolactone immobilized CPG in solution. The suspension of CPG in the working solution was continually mixed and incubated at 25° C. for 15 minutes. The CPG was allowed to settle and the supernatant was removed for analysis.

Galactose Competition Assay in 2× Matrix (or 5× Matrix or 1×PBS)

A 2688 nM (by mass) Alexa Fluor boronic acid dendrimer solution was prepared in 2× matrix (or 5× matrix or 1×PBS). A concentration dilution series of D-(+)-Galactose solutions, which included a range from 1,000,000×(2.688 M) to 0× (0 M) the mass of the boronic acid dendrimer, were prepared in 2× matrix (or 5× matrix or 1×PBS). 240 uL of a working solution containing 1 part 2.688 M galactose, 1 part 2688 nM Alexa Fluor boronic acid dendrimer and 2 parts 2× matrix (or 5× matrix or 1×PBS) was used to suspend 0.01 g of gluconolactone immobilized CPG in solution. The suspension of CPG in the working solution was continually mixed and incubated at 25° C. for 15 minutes. The CPG was allowed to settle and the supernatant was removed for analysis.

Data Analysis

The fluorescence intensities, again any detection system can be used, (650 nm/668 nm for Alexa Fluor 647) of supernatant aliquots were quantified on a fluorescence plate reader (Infinite M200, Tecan Inc., San Jose, Calif.). Analysis was done with the software package, Magellan Data Analysis Software. The fluorescent signals were analyzed by quantifying the intensity of each supernatant aliquot (40 uL with at least 3 replicate wells) and then using that data for analysis. Glucose (or fructose or galactose) competition curves were generated by plotting the concentration of D-(+)-Glucose (or D-(−)-Fructose or D-(+)-Galactose) versus the inverse of the free solution of fluorescence intensity measured during the assay (Fluorescence of DBA Bound to CPG (Bound)=Total Fluorescence Intensity of Solution—Fluorescence of Supernatant (Unbound)). FIGS. 21A to 21D shows a graphical representation of the intensity profile of glucose or other saccharide concentration. The data in FIG. 21A are the fluorescence intensity changes (Δl/l₀) of the G1+3-F-5-FPBA (I) dendrimer-boronic acid as a function of glucose concentration at 25 C.° in 2× plasma matrix at pH 7.4. The assay sensitivity as defined as the standard curve midpoint (IC₅₀) is approximately 10 mg/dl and has a slope with greater than or equal to a 2-log dynamic range. The data in FIG. 21B are the fluorescence intensity changes (Δl/l₀) of the G1+3-F-5-FPBA and G1+2-F-3-FPBA dendrimer-boronic acids as a function of glucose concentration at 25° C. in 2× plasma matrix at pH 7.4. The assay sensitivity as defined as the standard curve midpoint (IC₅₀) is approximately 10 mg/dl and 100 mg/dl and have slopes with greater than or equal to a 2-log dynamic range. The data in FIG. 21C are the fluorescence intensity changes (Δl/l₀) of the G1, G2, G3 and G4+3-F-5-FPBA dendrimer-boronic acids as a function of glucose concentration at 25 C.° in 2× plasma matrix at pH 7.4. The assay sensitivity as defined as the standard curve midpoint (IC₅₀) is approximately 10 mg/dl to >10,000 mg/dl. The data in FIG. 21D are the fluorescence intensity changes (Δl/l₀) of the G1+3-F-5-FPBA (I) dendrimer-boronic acid in response to the iDIOLgluconolactone modified CPG as a function of glucose, fructose and galactose concentration at 25 C.° in 2× plasma matrix at pH 7.4. Upon addition of fructose or galactose, the fluorescence intensity signal changed very little demonstrating that the DBA:IDIOL pair is minimally cross-reactive with the other hexoses.

### Quartz Crystal Signal Generator Experiment

A quartz crystal can be used as a signal generating device. A 5 MHz AT cut polished gold quartz crystal (1″ dia., Gold/Cr, Stanford Research Systems, Sunnyvale, Calif.) was cleaned using a piranha solution (3:1 mixture of concentrated sulfuric acid (H₂SO₄) and 30% aqueous hydrogen peroxide (H₂O₂) solution), ultra pure water and ethanol in series, and then dried by blowing a stream of nitrogen over surface of the crystal. The crystal was incubated in a solution of 1 mM 3-Aminopropanethiol (Sigma) (Note—other self-assembling monolayers were used in experiments) in anhydrous ethanol at 25° C. overnight. Following incubation, the gold surface was washed with ethanol and then ultra pure water and then dried by blowing a stream of nitrogen over the surface of the crystal. The crystal was incubated in a solution of 0.7 M gluconolactone (Sigma) in 85% DMA, 15% ultra pure water containing 1 mg mL⁻¹ DMAP at 25° C. overnight. Following incubation, the gold surface was washed with DMA and then ultra pure water and then dried by blowing a stream of nitrogen over the surface of the crystal. Any remaining amine sites of the immobilized SAM on the gold surface were blocked using a 1 M solution of succinic anhydride in DMF at 25° C. overnight. Following incubation, the gold surface was washed with DMF and then ultra pure water and then dried by blowing a stream of nitrogen over the surface of the crystal.

The modified gold quartz crystal was mounted in a crystal holder that is connected to the QCM25 Crystal Oscillator that was connected to the QCM200 Quartz Crystal Microbalance Digital Controller. A custom fit flow cell was attached to the holder. The flow cell/holder were fully immersed in a water bath at 35° C. Changes in resonance frequency and resistance were measured using the QCM200 Quartz Crystal Microbalance Digital Controller with an RS-232 communications port and software.

A gluconolactone modified crystal was mounted in the crystal holder. A 6-port injection valve connected to a pump was used to move buffer and/or reagents into the axial flow cell that was attached to the crystal holder. The system temperature was 35° C. Water was flowed into the cell and was monitored until a steady baseline was obtained. 70 uL of a 1344 nM solution of G1+3-F-5-FPBA (I) dendrimer-boronic acid in water was flowed into the cell. When the resonance frequency dropped and reached a stable value, the DBA bound to the gluconolactone (iDIOL) modified surface and reaction of the DBA with the surface was terminated. After the DBA was bound to the surface, water was flowed into the cell followed by a 242 mg/mL solution of glucose in water. When the resonance frequency increased and reached a stable value, the DBA unbound from the gluconolactone (iDIOL) modified surface and the reaction of the DBA with the glucose was terminated. A similar binding/unbinding cycle of the DBA (1344 nM aqueous solution of G1+3-F-5-FPBA (I)) to the iDIOL surface in the presence of glucose (2,421 mg/dL) was subsequently completed (See FIG. 23)

Response Time/On and Off Rate—The Rate/Time in which DBA Binds Off of Glucose and On to the iDIOL-CPG

A 2496 nM (by mass) Alexa Fluor® boronic acid dendrimer solution was prepared in matrix (for a final concentration of 624 nM). A concentration of D-(+)-Glucose, which included that found at the standard curve midpoint (IC₅₀)×4 was prepared in matrix (to give a final concentration of 4×IC₅₀ concentration). 240 μL of a working solution containing 1 part the 4×IC₅₀ concentration of D-(+)-Glucose, 1 part 2496 nM Alexa Fluor® boronic acid dendrimer, and 2 parts matrix were pipetted into a microcentrifuge tube. The microcentrifuge tube was allowed to incubate at 25° C. for 15 minutes while continually being mixed. After time, the working solution was added to a microcentrifuge tube containing 0.0100 g of iDIOL modified CPG and was continually mixed for 30 seconds. After time, CPG was spun down using a microarray high-speed centrifuge and supernatant aliquots removed for analysis. This was repeated for the following time points: 1, 1.5, 2, 2.5, 3, 3.5, 4, 4.5, 5, 5.5, 6, 8, 10, 12, 14 minutes. FIG. 22A show the relevant data. In FIG. 22A are data that show the response time or the time it takes the G1+2-F-3-FPBA dendrimer-boronic acid to reversibly bind on to the gluconolactone modified CPG and off of the glucose in the system whose concentration is at the IC₅₀ level in 2× matrix.

Response Time/On and Off Rate—The Rate/Time in which DBA Binds Off of the iDIOL-CPG and On to Glucose

A 2496 nM (by mass) Alexa Fluor® boronic acid dendrimer solution was prepared in matrix (for a final concentration of 624 nM). A concentration of D-(+)-Glucose, which included that found at the standard curve midpoint (IC₅₀)×4 was prepared in matrix (to give a final concentration of 4×IC₅₀ concentration). 180 μL of a working solution containing 1 part 2496 nM Alexa Fluor® boronic acid dendrimer and 2 parts matrix were pipetted into a microcentrifuge tube containing 0.0100 g of iDIOL modified CPG and was incubated at 25° C. while continually being mixed for 15 minutes. After time, a 60 uL solution of the 4×IC₅₀ concentration of D-(+)-Glucose was added to the contents of the micro centrifuge tube and continually mixed for 30 seconds. After time, the CPG was spun down using a microarray high-speed centrifuge and supernatant aliquots removed for analysis. This was repeated for the following time points: 1, 1.5, 2, 2.5, 3, 3.5, 4, 4.5, 5, 5.5, 6, 8, 10, 12, 14 minutes. FIG. 22B shows the relevant data. In FIG. 22B are data that show response time or the time it takes the G1+2-F-3-FPBA dendrimer-boronic acid to reversibly bind off of the gluconolactone modified CPG and on to the glucose in the system whose concentration is at the IC₅₀ level in 2× matrix.

Imaging and Data Analysis—Aliquots were scanned using a Tecan Infinite M200 Microplate Reader. Analysis was done with the Magellan Data Analysis Software. The fluorescent signals were analyzed by quantifying the intensity of each aliquot (unbound DBA) and then using the data for analysis. Response time/equilibrium curves were generated by plotting time versus the percentage of DBA bound to the iDIOL-CPG for each working solution. The amount of bound DBA was calculated by subtracting the amount of unbound DBA from the total amount of DBA in each system.

Affinity Chromatography Procedure—Solid-Phase Matrices Preparation—Synthesis of Polyol (Gluconolactone) Immobilization on Controlled Pore Glass (CPG) Chromatography Media

To 0.5 g of CPG-NH₂ (1000 Å, Millipore), add 3 mL of a 0.7 M solution of gluconolactone dissolved in a mixture containing 85% DMA, 15% HPLC grade water and 1 mg mL⁻¹ DMAP. Mix overnight on orbital shaker (170 rpm) at room temperature. Isolate modified CPG using vacuum filtration followed by three DMA and then three water washes. Unreacted amines were blocked by adding 3 mL of a 1 M solution of succinic anhydride in DMF to the 0.5 g batch of modified CPG. Mix overnight on orbital shaker (170 rpm) at room temperature. Isolate modified CPG using vacuum filtration followed by three DMF and then three water washes.

DBA Purification/Fractionation

Load DBA (fluorescently labeled with an Alexa Fluor tag or unlabeled) onto an affinity chromatography column packed with slurry of modified CPG prepared in 1×PBS or plasma fraction. Wash unreacted PAMAM and/or loosely bound DBA from the CPG by running 1×PBS or plasma fraction through column. Fractionate DBAs that are more tightly bound to the modified CPG by running increasing concentrations of glucose (0.5 mg/mL, 5 mg/mL, 50 mg/mL and 500 mg/mL) in 1×PBS or plasma fraction followed by increasing concentrations of gluconolactone (0.5 mg/mL, 5 mg/mL, 50 mg/mL, and 500 mg/mL) in 1×PBS or plasma fraction through column. Collect fractions and monitor the eluate by measuring fluorescence (excitation/emission dependent on Alexa Fluor tag) or absorbance at 360 nm, depending on whether the DBA is fluorescently labeled or not.

DBA Regeneration

Combine relevant fractions and change pH of eluate to 6 using 0.1 N HCl. Reduce volume of eluate to 1 mL. Load eluate containing DBA and glucose or gluconolactone onto a chromatography column packed with a slurry of size exclusion or gel filtration media prepared in 20 mM pH Ammonium Acetate, pH 6 (P2 Biogel, fine, BioRad). Separate fractionated DBA from glucose or gluconolactone molecules by running 20 mM Ammonium Acetate, pH 6 through column. Combine relevant fractions and neutralize solution using 0.1 N NaOH. Concentrate eluate by Speedvac to dryness. FIG. 24 shows the relevant data. FIG. 24 shows the fractionation of the G1+2-F-4-FPBA dendrimer-boronic acid in 1×PBS using an affinity column prepared with gluconolactone modified CPG.

The invention may suitably comprise, consist of, or consist essentially of, any of the disclosed or recited elements. The invention illustratively disclosed herein can be suitably practiced in the absence of any element which is not specifically disclosed herein. The various embodiments described above are provided by way of illustration only and should not be construed to limit the claims attached hereto. It will be recognized that various modifications and changes may be made without following the example embodiments and applications illustrated and described herein, and without departing from the true spirit and scope of the following claims.

1-26. (canceled)

27. A system for detecting an organic analyte, the system comprising:
(a) a surface comprising an immobilized polyol with at least two hydroxyl groups; and
(b) a dendrimer-boronic acid compound
wherein the dendrimer-boronic acid compound is reversibly associated with an immobilized polyol with affinity constant K_{id} selected such that the presence of an analyte having an affinity constant (K_{ad}) between the dendrimer-boronic acid causes competition between the analyte and the immobilized polyol and the degree of competition is proportional to the concentration of the analyte, and the system produces a signal proportional to the concentration of the analyte.

28. The system of claim 27 wherein the dendrimer comprises a group that produces a detectable signal when the dendrimer-boronic acid compound is released from the immobilized polyol.

29. The system of claim 27 wherein the dendrimer of the dendrimer-boronic acid compound comprises a PAMAM dendrimer.

30. The system of claim 27 where the dendrimer-boronic acid compound comprises a boronic acid compound comprising the structure: 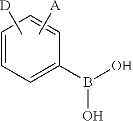
wherein D comprises a dendrimer group and A comprises a group containing an oxygen, sulfur, amino, imino, or alkoxy; including such groups as hydrogen, halogen (such as F— and Cl—), —CHO, —OH, —SH, —NH₂, —NHR₁, —N(R₁)₂, —CO₂H, —CO₂ R₁, —CO—NH₂, —CO—NH—R₁, —CO—N(R₁)₂, —CONH—NH₂, and R₁ is independently alkyl of from 1 to 5 carbon atoms.

31. The system of claim 27 wherein the analyte is glucose.

32. The system of claim 27 where the dendrimer-boronic acid compound comprises a dendrimer-boronic acid compound comprising the compound: 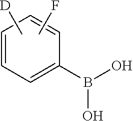
wherein F is a fluorine moiety and D is a dendrimer.

33. The system of claim 27 wherein the surface comprises a metallic, glass or a thermoplastic polymeric surface.

34. The system of claim 27 wherein the system comprises a mechanical electrical or chemical detector that produces a signal proportional to the analyte concentration.

35. The system of claim 33 wherein the system comprises a remote signal receiver that can detect the signal proportional to the analyte concentration.

36. The system of claim 27 wherein the system comprises at least a portion of a microcantilever structure having a resident frequency such that a change in mass of the cantilever changes the resident frequency in proportion to the change in mass.

37. The system of claim 27 wherein the system comprises at least a portion of a crystal structure having a resident frequency such that a change in mass of the crystal changes the resident frequency in proportion to the change in mass.

38. A sensor for an organic analyte, the sensor comprising:
(a) a container permeable to the analyte;
(b) held within the container, a detector producing a signal proportional to the analyte concentration within the container; and
(c) a signal receiver, remote from the container, that can detect the signal proportional to the analyte concentration.

39. The sensor of claim 38 wherein the detector comprises a surface having an immobilized polyol with at least two hydroxyl groups, and reversibly bonded to the immobilized polyol, a dendrimer-boronic acid compound, wherein the dendrimer-boronic acid compound is reversibly associated with an immobilized polyol with affinity constant K_{id} selected such that the presence of an analyte having an affinity constant (K_{ad}) causes the analyte to compete with the immobilized polyol.

40. The sensor of claim 38 wherein the detector comprises a cantilever or crystal.

41. The sensor of claim 39 where the dendrimer-boronic acid compound comprises a dendrimer-boronic acid compound comprising the structure: 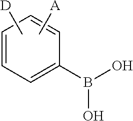
wherein D comprises a dendrimer group and A comprises a group containing an oxygen, sulfur, amino, imino, or alkoxy; including such groups as hydrogen, halogen (such as F— and Cl—), —CHO, —OH, —SH, —NH₂, —NHR₁, —N(R₁)₂, —CO₂H, —CO₂ R₁, —CO—NH₂, —CO—NH—R₁, —CO—N(R₁)₂, —CONH—NH₂, and R₁ is independently alkyl of from 1 to 5 carbon atoms.

42. The sensor of claim 27 wherein dendrimer also comprises a group that produces a detectable signal comprising a RF signal when the boronic acid is released from the immobilized polyol in proportion to analyte concentration.

43. The sensor of claim 27 where the dendrimer-boronic acid compound comprises a compound selected from the group of: 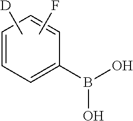
wherein F is a fluorine moiety and D is a dendrimer.

44. The sensor of claim 38 wherein the surface comprises a metallic, glass or a thermoplastic polymeric surface.

45. The system of claim 27 wherein the dendrimer or dendrimer-boronic acid component is a fraction of an original reaction product produced by a separation of dendrimer or dendrimer-boronic acid components that differ by molecular weight, molecular diameter or number of functional groups.

46. The system of claim 27 wherein the dendrimer or dendrimer-boronic acid component is a fraction characterized by its number of boronic acid functional groups and the resulting affinity constants (K_{ad} and K_{id}) that are specific to that fraction and determine the degree by which the dendrimer-boronic acid component can compete with the analyte and the immobilized polyol.
